(19) United States
(12) Patent Application Publication
(10) Pub. No.: US 2023/0174598 A1
(43) Pub. Date: Jun. 8, 2023

(54) MODIFIED ARRESTIN-1 TO ENHANCE PHOTORECEPTOR SURVIVAL IN RETINAL DISEASE

(71) Applicant: UNIVERSITY OF FLORIDA RESEARCH FOUNDATION, INCORPORATED, Gainesville, FL (US)

(72) Inventors: Wesley Clay SMITH, Gainesville, FL (US); Astra DINCULESCU, Gainesville, FL (US); Vince A. CHIODO, Gainesville, FL (US)

(21) Appl. No.: 17/906,098

(22) PCT Filed: Mar. 18, 2021
(86) PCT No.: PCT/US2021/022899

§ 371 Date: Sep. 12, 2022

Related U.S. Application Data
(60) Provisional application No. 62/991,244, filed on Mar. 18, 2020.

(51) Int. Cl. C07K 14/47; A61K 48/00; A61P 27/02

(52) U.S. Cl. CPC C07K 14/4703; A61K 48/005; A61K 48/0075; A61P 27/02

(57) ABSTRACT

Arrestin-1 variants having reduced inhibitory effects on enolase-1 catalytic activity are described. The arrestin-1 variants can be used in the treatment of retinal degenerative diseases.

### CROSS-REFERENCE TO RELATED APPLICATIONS

This application claims the benefit of U.S. Provisional Application No. 62/991,244, filed Mar. 18, 2020, which is incorporated herein by reference.

### SEQUENCE LISTING

The Sequence Listing written in file 555774_SeqList_ST25.txt is 104 kilobytes in size, was created Mar. 5, 2021, and is hereby incorporated by reference

### INTRODUCTION

The arrestin family of proteins is a key regulator of G protein-coupled receptors (GPCRs), functioning in both desensitization and intracellular trafficking of GPCRs. In the vertebrate visual system, arrestin-1 desensitizes the visual pigment in both rod and cone photoreceptors, binding light-activated and phosphorylated rhodopsin to sterically occlude transducin. In addition to its interaction with rhodopsin, arrestin-1 interacts with additional cellular components including calcium-calmodulin, Src family tyrosine kinases, E3 ubiquitin ligases; and N-ethylmaleimide sensitive factor (NSF).

In addition to these binding partners, arrestin-1 also interacts with enolase-1 (Smith et al. (2011) Interaction of arrestin with enolase1 in photoreceptors. Invest Ophthalmol Vis Sci 52, 1832-1840). Enolase-1, which catalyzes the interconversion of 2-phosphoglycerate to phosphoenolpyruvate, is one of the key enzymes in the glycolysis pathway. Binding of arrestin-1 to enolase-1 affects the catalytic activity of enolase, reducing its rate of activity by ˜25%. Photoreceptors require about 10⁵ ATP molecules per second per cell. Further, photoreceptors metabolize 80-96% of available glucose into lactic acid via aerobic glycolysis (Kanow et al. (2017) Biochemical adaptations of the retina and retinal pigment epithelium support a metabolic ecosystem in the vertebrate eye. eLife 6, e28899 and Hurley et al. (2015) Glucose, lactate, and shuttling of metabolites in vertebrate retinas. J Neurosci Res 93, 1079-1092). The lactate byproduct is an essential metabolic component to the retinal pigmented epithelium and Mller glia. Because of the extreme energy demands of photoreceptors, small changes in glycolytic efficiency could have a large impact.

### SUMMARY

Described are arrestin-1 variants that have reduced inhibitory effect on enolase-1 catalytic activity while retaining binding of enolase-1.

We have investigated interaction between arrestin-1 and enolase-1 and mapped the molecular contacts between the two proteins. Using this map, we identified how the binding of arrestin-1 affects the catalytic activity of enolase-1. Using fluorescence quench protection of strategically placed fluorophores on the arrestin-1 surface, our study identified that arrestin-1 primarily engages enolase-1 along the surface opposite the side of the molecule that binds photo-activated rhodopsin. Using this information, we identified amino acids positions in arrestin-1 that, when mutated, reduced the inhibitory effect of arrestin-1 on enolase-1 catalytic activity.

In some embodiments, the arrestin-1 variants inhibit enolase-1 catalytic activity by less than 25%, less than 20%, less than 19%, less than 18%, less than 17%, less than 16%, less than 15%, less than 14%, less than 13%, less than 12%, less than 110, less than 10%, less than 9%, less than 8%, less than 7%, less than 6%, less than 5%, less than 4%, less than 3%, less than 2%, or less than 1%, relative to the activity of enolase-1 in the absence of arrestin-1.

In some embodiments, the arrestin-1 variants inhibit enolase-1 catalytic activity by less than 50%, less than 45%, less than 40%, less than 35%, less than 30%, less than 25%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1%, when comparing inhibition of enolase-1 catalytic activity by the arrestin-1 variant relative to inhibition of enolase-1 catalytic activity by wild-type arrestin-1. In some embodiments, the arrestin-1 variants do not substantially inhibit enolase catalytic activity.

In some embodiments, the arrestin-1 variants do not substantially affect binding of arrestin-1 to enolase-1. The arrestin-1 variants are able to interact with enolase-1 at a level similar to wild-type arrestin-1 as determined by immunoprecipitation pulldown assay.

Also described are nucleic acids and expression vectors encoding the described arrestin-1 variants.

The described arrestin-1 variants can be delivered to the eye using known delivery and gene therapy methods or vectors. Gene therapy methods include, but are not limited to, naked DNA delivery, RNA delivery, non-viral DNA/RNA delivery, viral delivery, and electroporation. Viral delivery methods include, but are not limited to, adeno-associated virus (AAV)-based delivery methods, adenovirus-based delivery methods, and lentivirus-based delivery methods.

In some embodiments, a vector encoding an arrestin-1 variant is administered by subretinal injection. In some embodiments, a vector encoding an arrestin-1 variant is administered by intravitreal injection. In some embodiments, a vector encoding an arrestin-1 variant is administered by suprachoroidal injection.

The described arrestin-1 variants and nucleic acids encoding the arrestin-1 variants can be used in the treatment of retinal degenerative diseases in a subject. Treatment comprises administering to the retina of the subject a described arrestin-1 variant or a nucleic acid encoding a described arrestin-1 variant. In some embodiments, the described arrestin-1 variants can be used in the treatment of retinal degenerative diseases that are associated with loss of rod and/or cone photoreceptors. Retinal diseases that can be treated using the arrestin-1 variants include, but are not limited to, retinitis pigmentosa, cone-rod dystrophies, and Usher's syndromes. In some embodiments, described arrestin-1 variants and nucleic acids encoding the arrestin-1 variants can be used to treat a subject suffering from, or at risk of suffering from retinal degeneration, retinitis pigmentosa, cone-rod dystrophies, or Usher's syndromes. In some embodiments, the described arrestin-1 variants and nucleic acids encoding the arrestin-1 variants can be used to treat at least one symptom associated with retinal degeneration, retinitis pigmentosa, cone-rod dystrophies, or Usher's syndromes. In some embodiments, the described arrestin-1 variants and nucleic acids encoding the arrestin-1 variants can be used to decrease, prevent, or delay onset of retinal degeneration, or decrease, prevent, or delay onset of vision loss, in a subject suffering from, or at risk of suffering from retinitis pigmentosa, cone-rod dystrophies, or Usher's syndromes. In some embodiments, the described arrestin-1 variants and nucleic acids encoding the arrestin-1 variants can be used to increase survival of photoreceptors and/or improve photoreceptor function in a subject suffering from, or at risk of suffering from retinal degeneration, retinitis pigmentosa, cone-rod dystrophies, or Usher's syndromes. In some embodiments, the arrestin-1 variants can be delivered to a retina to decrease degradation of photoreceptors.

In some embodiments, expression of a nucleic acid encoding a described arrestin-1 variant in a retina protects photoreceptors against degeneration. In some embodiments, expression of a nucleic acid encoding a described arrestin-1 variant in rods and/or cones increases the rate of glycolysis in the rods and/or cones. In some embodiments, expression of a nucleic acid encoding a described arrestin-1 variant in rods and/or cones preserves scotopic and/or photopic vision in a subject having a retinal degenerative disease or a subject susceptible to retinal degeneration. In some embodiments, expression of a nucleic acid encoding a described arrestin-1 variant in rods and/or cones preserves scotopic and/or photopic vision as determined by electroretinography (ERG) in a subject having a retinal degenerative disease or a subject susceptible to retinal degeneration. In some embodiments, expression of a nucleic acid encoding a described arrestin-1 variant in rods and/or cones in a subject having a retinal degenerative disease or a subject susceptible to retinal degeneration decreases degeneration of the rods and/or the cones. In some embodiments, expression of a nucleic acid encoding a described arrestin-1 variant in rods and/or cones in a subject having a retinal degenerative disease or a subject susceptible to retinal degeneration decreases loss of scotopic and/or photopic vision. In some embodiments, expression of a nucleic acid encoding a described arrestin-1 variant in rods and/or cones increases survival of photoreceptors. In some embodiments, expression of a nucleic acid encoding a described arrestin-1 variant in rods and/or cones in a subject having a retinal degenerative disease or a subject susceptible to retinal degeneration improves photoreceptor function, decreases degeneration of the rods and/or cones, decreases loss of scotopic and/or photopic vision, and/or increases survival or photoreceptors.

The subject having a retinal degenerative disease can be a mammal. The mammal can be, but is not limited to, a mouse, a rat, a guinea pig, a rabbit, a human, a non-human primate, a dog, a cat, a bovine, and a sheep.

### BRIEF DESCRIPTION OF THE FIGURES

FIGS. 1A-1B. Graph illustrating optimization of fluorescence quench and quench protection. FIG. 1A) Potassium iodide-induced quenching of three different cysteine-substituted mutations of arrestin-1—K53C, S86C, and Y125C—labeled with monobromobimane (mBBr); 3 μM arrestin-1-bimane was mixed with increasing concentration of potassium iodide (KI) and fluorescence emission of the mBBr measured (n=3; curve shows best fit of non-linear regression analysis). FIG. 1B) Protection of KI-induced quenching of H10C-mBBr by enolase-1; 0.25 μM H10C-mBBr was mixed with increasing concentration of enolase-1 (0-12.5 μM) in the presence of 100 mM KI, and fluorescence emission of the mBBr measured; dashed line indicates H10C-mBBr fluorescence with no KI present. Inset shows the fluorescent emission curves of mBBr-labeled arrestin-1 with increasing concentrations of enolase-1 (0 μM+KI, 0.25 μM, 1.25 μM, 2.5 μM, 5 μM, and 12.5 μM Enolase-1; x-axis=wavelength in nm; y-axis=fluorescence (cps)); the blood trace shows the fluorescence of H10C-mBBr with no KI present.

FIGS. 2A and 2B. Protection of fluorescence quenching of bimane-labeled arrestin-1 by enolase-1. FIG. 2A) Various cysteine-substituted mutants of arrestin-1 were labeled with bimane and quenching of fluorescence measured by potassium iodide in the presence or absence of 20-fold molar excess of enolase-1 (each bar shows mean+/−SEM (n=4). Mutants for which enolase provided >75% protection of the KI quenching are shown in dark gray, 50-75% in medium gray, 25-49% in light gray, and 0-24% in very light gray. FIG. 2B) The various mutants indicated in “A” are shown plotted on a three-dimensional rendering of arrestin-1, retaining the same color coding for amino acids as in “A”; the four models show four views of the same rendering (two sides, top, and bottom).

FIGS. 3A and 3B. Molecular model illustrating arrestin-1 (dark gray) docked with a dimer of enolase-1 (light gray). FIG. 3A) Docking model showing opposite sides of the same model, with arrestin-1 residues His-10, Asp-183, Glu-218, Glu-302, and Asp-362 indicated in black. FIG. 3B) Magnified view of the interface area as shown in FIG. 3A.

FIGS. 4A-4C. Enolase-1 pulldown by mutants of arrestin-1 designed to disrupt charge-pair interactions with enolase-1. FIG. 4A) Molecular model of the arrestin-1/enolase-1 complex showing the charged residues on arrestin-1 (ball and stick) that were selected for reversal, and their proximity to enolase-1 (surface representation, with enolase-1 charged pair shown). FIG. 4B) arrestin-1 with the indicated point mutations were immunoprecipitated with an anti-arrestin monoclonal antibody attached to magnetic beads, pulling down fluorescently labelled enolase-1. The captured enolase-1 is normalized to the pulldown of enolase-1 by arrestin-1 with no mutations (WT); the bar indicated as “no Arr” shows the background pulldown of labeled enolase-1 in the absence of any arrestin-1. FIG. 4C) enolase-1 pulldown with arrestin-1 with all seven point mutations (SDM-Arr) or arrestin-1 pulldown of enolase-1 with all seven charge-pair mutation (SDM-Eno) showed essentially no pulldown. Combining SDM-Arr with SDM-Eno restored pulldown of enolase. enolase-1 pulldown by the mutant arrestins that are significantly different than WT are indicated with an asterisk (ρ<0.05).

FIGS. 5A-5C. Arr-E361G/D362G binds enolase-1, but does not affect enolase catalytic activity. FIG. 5A) Molecular model of the Arr1-Enol complex showing the proximity of Glu-361/Asp-362 on Arr1 relative to active site loops L1, L2, and L3 on Enol. FIG. 5B) Immunoprecipitation pulldown of fluorescently labelled enolase-1 was performed using native arrestin-1 (Arr-WT) or arrestin-1 with E361G and D362G mutations (Arr-GG). The binding of enolase-1 was not significantly changed by the double mutations E361G/D362G; bars show means+/−SEM (n=8). FIG. 5C) The influence of these same two mutations on enolase-1 catalytic activity was assessed, monitoring the production of ATP from the processing of 2-phosphoglycerate to pyruvate (enolase catalyzes the reaction of 2-phosphoglycerate to phosphoenolpyruvate, which is then converted to pyruvate with the production of ATP). The E361G/D362G arrestin-1 (closed circles) did not show inhibition of enolase-1 activity that is evident with native arrestin-1 (open squares); each point shows mean+/−SEM (n=3).

FIG. 6. Graph illustrating effect of amino acid substitution at positions 361 and 362 on arrestin inhibition of enolase catalytic activity.

FIGS. 7A-7C. Retinal flatmounts from wild-type C57bl/6j mice injected with wild type (WT) arrestin or GEAr1 arrestin (E361G/D362G) and visualized with two-photon microscopy to image levels of NADH (reduced nicotinamide adenine dinucleotide)(FIG. 7A). Graphs illustrating NADH levels in retinas from mice injected with wild-type (Arr) or GEAr1 arrestin (E361G/D362G) (FIG. 7B) or ATP levels in homogenates of the retinas (FIG. 7C).

FIG. 8. Optical coherence tomography of the retinal layers in C57bl/6j mice.

FIG. 9. Graphs illustrating optical coherence tomography at days 20 and 120 in wild-type mice and mice treated with GEAr1 arrestin.

FIGS. 10A-10C. Graphs illustrating rod and cone response in retinas from P23H mice treated with buffer or GEAr1 arrestin

FIG. 11. Graphs illustrating rod and cone response in retinas from P23H mice treated with buffer or GEAr1 arrestin.

FIGS. 12A-12C. Graphs illustrating photoreceptor survival in the untreated and GEAr1-treated P23H mice showing increase survival of rod and cone cell bodies (FIGS. 12A and 12B) and increased NADH and ATP levels in GEAr1 arrestin treated P23H mice (FIG. 12C).

FIG. 13. Graphs illustrating in P23 Mouse model of Retinitis Pigmentosa treated with wild-type arrestin (Arr1).

FIG. 14A. Schematic for GEAr1 and ArrWT design for packaging into AAV.

FIG. 14B. Graph illustrating lactate secretion rate derivation from retinas treated with AAV-GEAr1 or buffer-injected controls.

FIG. 14C. Graph illustrating increased lactate secretion in retinas treated with AAV-GEAr1 compared with retinas treated with AAV-ArrWT or injected with buffer (ρ<0.05).

FIG. 14D. Graph illustrating survival of photoreceptors as measured by outer nuclear layer thickness in C57BL/6 mice expression either GEAr1 or ArrWT 6 months after injection.

FIG. 15. Graphs illustrating scotopic and photopic ERG function in P23H+/− mice treated subretinally with AAV-GEAr1 or AAV-ArrWT. ERG function was measured 30 days after injection using a Celeris ERG system. Samples that are statistically different than a sham-injected eye are shown (ρ values as indicated. Student's paired t-test).

FIG. 16A. Graph illustrating expression levels of GEAr2 in retina following intravitreal injection of the indicated AAV serotypes packaged with hGRK1-myc-GEAr1-2A-GFP (one-way ANOVA, ρ<0.05).

FIG. 16B. Graphs illustrating scotopic and photopic ERG function following intravitreal injection of the indicated AAV serotypes packaged with hGRK1-myc-GEAr1-2A-GFP (one-way ANOVA, ρ>0.05).

FIG. 17A. Graph illustrating expression levels of GEAr1, relative to native arrestin-1, in mouse eyes expressing myc-GEAr1 driven by the indicated promoter (one-way ANOVA, ρ0.05).

FIG. 17B. Graph illustrating time course of photoreceptor function (scotopic and photopic ERG function) and survival in P23H+/− mice retinas treated with GEAr1 driven by hOps promoter (*, Student's t-test, ρ<0.05).

FIG. 17C. Graph illustrating time course of photoreceptor function (scotopic and photopic ERG function) and survival in P23H+/− mice retinas treated with GEAr1 driven by smCBA promoter (*, Student's t-test, ρ<0.05).

### DESCRIPTION

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions such as “not including the endpoints”; thus, for example, “within 10-15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s) or method step(s) that do not materially change the basic and novel characteristics of the compositions and methods described herein may be included in those compositions or methods.

A “nucleic acid” includes both RNA and DNA. RNA and DNA include, but are not limited to, cDNA, genomic DNA, plasmid DNA, synthetic RNA or DNA, and mRNA. A nucleic acid can be formulated with a delivery agent such as, but not limited, a cationic lipid, a peptide, a cationic polymer, or a virus. Nucleic acid also includes modified RNA or DNA.

A “vector” comprises a nucleic acid sequence encoding an expression product (e.g., a peptide (i.e., polypeptide or protein such as any of the described arrestin-1 variants) or an RNA). An expression product can be an arrestin-1 variant. A vector can be, but is not limited to, a plasmid, viral vector, a construct, or composition comprising a nucleic acid encoding the expression product. A vector may comprise one or more sequence that facilitate replication of a sequence in a cell and/or integration of a sequence into a target nucleic acid sequence. A vector may comprise one or more sequences necessary for expression of the encoded expression product in a cell. The vector may comprise one or more of: an enhancer, a promoter, intron, a terminator, and a polyA signal operably linked to the DNA coding sequence. A vector may also comprise one or more sequences that alter stability of a messenger RNA (mRNA), RNA processing, or efficiency of translation. A viral vector can be, but is not limited to, an adeno-associated virus (AAV). Vectors can be manufactured in large scale quantities and/or in high yield. Vectors can be manufactured using GMP manufacturing. Vectors are can be formulated to be safe and effective for injection into a mammalian subject.

“Protein,” “peptide,” or “polypeptide” includes a contiguous string of two or more amino acids. A “protein sequence,” “peptide sequence,” “polypeptide sequence,” or “amino acid sequence” refers to a series of two or more amino acids in a protein, peptide, or polypeptide.

“Operably linked” refers to the juxtaposition of two or more components (e.g., a promoter and another sequence element) such that both components function normally and allow the possibility that at least one of the components can mediate a function that is exerted upon at least one of the other components. For example, a promoter operably linked to a coding sequence will direct RNA polymerase-mediated transcription of the coding sequence into RNA, including mRNA, which may then be spliced (if it contains introns) and, optionally, translated into a protein encoded by the coding sequence. A coding sequence can be “operably linked” to one or more transcriptional or translational control sequences. A terminator/polyA signal operably linked to a gene terminates transcription of the gene into RNA and directs addition of a polyA signal onto the RNA.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, chicken β-actin promoter, modified chicken β-actin promoter (smCBA), opsin promoter, human opsin promoter, truncated human opsin promoter (hOps), rhodopsin kinase promoter, human rhodopsin kinase promoter, Igκ promoter, mPGK, SV40 promoter, β-actin promoter, α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EF1α promoter. The CMV promoter can be, but is not limited to, CMV immediate early promoter, human CMV promoter, mouse CNV promoter, and simian CMV promoter. In some embodiments, the promoter is a retina-specific promoter.

“Arrestin-1” (sometimes referred to as S-antigen, visual arrestin or rod arrestin), is an arrestin-family member responsible for inactivation of the G protein-coupled receptor rhodopsin in photoreceptors. Arrestin-1 is expressed at high levels in both rod and cone photoreceptor cells. Arrestin-1 also interacts with additional protein partners, including enolase-1, and affects other signaling cascades beyond phototransduction.

“Enolase-1” (alpha-enolase) is a glycolytic enzyme that catalyzes the conversion of 2-phosphoglycerate to phosphoenolpyruvate.

“Orthologs” are genes and products thereof in different species that evolved from a common ancestral gene by speciation and retain the same or similar function. An ortholog is a gene that is related by vertical descent and is responsible for substantially the same or identical functions in different organisms. For example, mouse arrestin-1 and human arrestin-1 can be considered orthologs. Genes may share sequence similarity of sufficient amount to indicate they are orthologs. Protein may share three-dimensional structure of sufficient amount to indicate the proteins and the genes encoding them are orthologs. Methods of identifying orthologs are known in the art.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters or by inspection and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

Amino acid substitution indicates that an amino acid normally present at a given position in a protein amino acid sequence is mutated to a different amino acid. For instance, an aspartate residue at position 362 corresponding to SEQ ID NO: 30 can be mutated to glycine. In this example there is an amino acid substitution (or simply a substitution) of an aspartate for a glycine at position 362. Substitutions can be made using any of the methods known in the art. Amino acid substitutions can alter the activity of the protein, e.g., the glycine and/or alanine substitutions in the arrestin-1 variants described herein. Alternatively, amino acid substitutions can have little to no effect of activity or interactions of a protein.

In the present description and claims, the conventional one-letter and three-letter codes for amino acid residues are used. E361G indicates the glutamate (or glutamic acid) residue at position 361 (corresponding to SEQ ID NO: 30) is changed to glycine. E361G/A indicates the glutamate (or glutamic acid) residue at position 361 (corresponding to SEQ ID NO: 30) is changed to glycine or alanine.

The terms “treat,” “treatment,” and the like, mean the methods or steps taken to provide relief from or alleviation of the number, severity, and/or frequency of one or more symptoms of a disease or condition in a subject.

A “pharmacologically effective amount,” “therapeutically effective amount,” “effective amount,” or “effective dose” refers to that amount of an agent to produce the intended pharmacological, therapeutic, or preventive result.

### Arrestin-1 Variants

Described are arrestin-1 variants that have reduced inhibitory effect on enolase-1 catalytic activity while retaining binding of enolase-1. The arrestin-1 variants comprise amino acid substitutions of the glutamate residue and/or the aspartate residue at amino acid positions in arrestin-1 corresponding to positions 361 and 362, respectively, of SEQ ID NO: 30 (bovine arrestin-1), wherein the substitution at each position is independently alanine or glycine. The arrestin-1 can be a bovine arrestin or an ortholog thereof. The glutamate and aspartate residues may not be in the exact same numerical positions in arrestin-1 orthologs. For example, the glutamate and aspartate residues that correspond to the glutamate and aspartate at positions 361 and 362, respectively, of SEQ ID NO: 30 occur at positions 362 and 363 of the mouse ortholog (SEQ ID NO: 4) and at positions 365 and 366 of the human ortholog (SEQ ID NO: 18). In some embodiments, the ortholog is a mammalian ortholog. The mammalian ortholog can be, but is not limited to, a mouse arrestin-1, a rat arrestin-1, a guinea pig arrestin-1, a rabbit arrestin-1, a human arrestin-1, a non-human primate arrestin-1, a dog arrestin-1, a cat arrestin-1, or a sheep arrestin-1. In some embodiments, the arrestin-1 can be a human arrestin-1.

In some embodiments, the arrestin-1 variants inhibit enolase-1 catalytic activity by less than 25%, less than 20%, less than 19%, less than 18%, less than 17%, less than 16%, less than 15%, less than 14%, less than 13%, less than 12%, less than 110, less than 10%, less than 9%, less than 8%, less than 7%, less than 6%, less than 5%, less than 4%, less than 3%, less than 2%, or less than 1%, relative to the activity of enolase-1 in the absence of arrestin-1.

In some embodiments, the arrestin-1 variants inhibit enolase-1 catalytic activity by less than 50%, less than 45%, less than 40%, less than 35%, less than 30%, less than 25%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1%, when comparing inhibition of enolase-1 catalytic activity by the arrestin-1 variant relative to inhibition of enolase-1 catalytic activity by wild-type arrestin-1. In some embodiments, the arrestin-1 variants do not substantially inhibit enolase catalytic activity. Less inhibition indicates higher enolase-1 catalytic activity, e.g., inhibiting enolase-1 activity by less than 20% indicates enolase-1 retains greater than 80% activity.

In some embodiments, the inhibition of enolase-1 catalytic activity by an arrestin-1 variant is than 50%, less than 45%, less than 40%, less than 35%, less than 30%, less than 25%, 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the inhibition of enolase-1 catalytic activity by wild-type arrestin-1.

In some embodiments, the arrestin-1 variants do not substantially affect binding of arrestin-1 to enolase-1. Binding of enolase-1 by arrestin-1 can be measured by immunoprecipitation pulldown assay or by other assays known in the art for measuring protein-protein interaction. In some embodiments, the arrestin-1 variants are able to interact with enolase-1 at a level similar to wild-type arrestin-1 as determined by immunoprecipitation pulldown assay. In some embodiments, arrestin-1 variant binding to enolase-1 as measured in an immunoprecipitation pulldown assay is at least 80%, at least 85%, at least 90%, or at least 95% of wild-type arrestin-1 binding to enolase-1 as measured in the immunoprecipitation pulldown assay.

In some embodiments, the arrestin-1 variant contains an alanine or glycine substitution of the glutamate residue at position 361 corresponding to SEQ ID NO: 30, i.e., E361A arrestin-1 or E361G arrestin-1. In some embodiments, the arrestin-1 variant contains a glycine substitution at position 361 corresponding to SEQ ID NO: 30, i.e., E361G arrestin-1. In some embodiments, the arrestin-1 variant contains an alanine substitution at position 361 corresponding to SEQ ID NO: 30, i.e., E361A arrestin-1.

In some embodiments, the arrestin-1 variant contains an alanine or glycine substitution of the glutamate residue at position 365 corresponding to SEQ ID NO: 18, i.e., E365A arrestin-1 or E365G arrestin-1. In some embodiments, the arrestin-1 variant contains a glycine substitution at position 365 corresponding to SEQ ID NO: 18, i.e., E365G arrestin-1. In some embodiments, the arrestin-1 variant contains an alanine substitution at position 366 corresponding to SEQ ID NO: 18, i.e., E366A arrestin-1.

In some embodiments, the arrestin-1 variant contains an alanine or glycine substitution of the aspartate residue at position 362 corresponding to SEQ ID NO: 30, i.e., D362A arrestin-1 or D362G arrestin-1. In some embodiments, the arrestin-1 variant contains a glycine substitution at position 362 corresponding to SEQ ID NO: 30, i.e., D362G arrestin-1. In some embodiments, the arrestin-1 variant contains an alanine substitution at position 362 corresponding to SEQ ID NO: 30, i.e., D362A arrestin-1.

In some embodiments, the arrestin-1 variant contains an alanine or glycine substitution of the aspartate residue at position 366 corresponding to SEQ ID NO: 18, i.e., D366A arrestin-1 or D366G arrestin-1. In some embodiments, the arrestin-1 variant contains a glycine substitution at position 366 corresponding to SEQ ID NO: 18, i.e., D366G arrestin-1. In some embodiments, the arrestin-1 variant contains an alanine substitution at position 366 corresponding to SEQ ID NO: 18, i.e., D366A arrestin-1.

In some embodiments, the arrestin-1 variant contains alanine substitutions of the glutamate residue at position 361 and the aspartate residue at position 362 corresponding to SEQ ID NO: 30, i.e., E361A/D362A arrestin-1. In some embodiments, the arrestin-1 variant contains glycine substitutions of the glutamate residue at position 361 and the aspartate residue at position 362 corresponding to SEQ ID NO: 30, i.e., E361G/D362G arrestin-1 (GEAr1 arrestin). In some embodiments, the arrestin-1 variant contains an alanine substitution of the glutamate residue at position 361 and a glycine substitution of the aspartate residue at position 362 corresponding to SEQ ID NO: 30, i.e., E361A/D362G arrestin-1. In some embodiments, the arrestin-1 variant contains a glycine substitution of the glutamate residue at position 361 and an alanine substitution of the aspartate residue at position 362 corresponding to SEQ ID NO: 30, i.e., E361G/D362A arrestin-1.

In some embodiments, the arrestin-1 variant contains alanine substitutions of the glutamate residue at position 365 and the aspartate residue at position 366 corresponding to SEQ ID NO: 18, i.e., E365A/D366A arrestin-1. In some embodiments, the arrestin-1 variant contains glycine substitutions of the glutamate residue at position 365 and the aspartate residue at position 366 corresponding to SEQ ID NO: 18, i.e., E365G/D366G arrestin-1 (GEAr1 human arrestin). In some embodiments, the arrestin-1 variant contains an alanine substitution of the glutamate residue at position 365 and a glycine substitution of the aspartate residue at position 366 corresponding to SEQ ID NO: 18, i.e., E365A/D366G arrestin-1. In some embodiments, the arrestin-1 variant contains a glycine substitution of the glutamate residue at position 365 and an alanine substitution of the aspartate residue at position 366 corresponding to SEQ ID NO: 18, i.e., E365G/D366A arrestin-1.

In some embodiments, an arrestin-1 variant having a glycine or alanine substitution at positions corresponding to positions 361 and/or 362 of SEQ ID NO: 30, has reduced inhibitory effect on enolase-1 catalytic activity compared to wild-type arrestin-1, is able to bind to enolase-1, and comprises an amino acid sequence that is at least 70%, at least 75%, at least 80%, at least 85%, at least 90%, or at least 95% identical to SEQ ID NO: 30.

In some embodiments, an arrestin-1 variant having a glycine or alanine substitution at positions corresponding to positions 365 and/or 366 of SEQ ID NO: 18, has reduced inhibitory effect on enolase-1 catalytic activity compared to wild-type arrestin-1, is able to bind to enolase-1, and comprises an amino acid sequence that is at least 70%, at least 75%, at least 80%, at least 85%, at least 90%, or at least 95% identical to or 100% identical to any of SEQ ID NO: 21-28.

In some embodiments, an arrestin-1 variant has an amino acid sequence comprising the amino acid sequence of SEQ ID NO: 6, 9-16, or 21-28, or an ortholog thereof. In some embodiments, an arrestin-1 variant has an amino acid sequence comprising the amino acid sequence of SEQ ID NO: 6, 9-16, or 21-28 having 1, 2, 3, 4, 5, 6, 7, 8, 9, or 10 amino acid substitutions at amino acid positions other than at positions corresponding to positions 361 and 362 of SEQ ID NO: 30, wherein the substitutions do no adversely affect the activity of the arrestin-1.

### Nucleic Acids Encoding Arrestin-1 Variants

Nucleic acids encoding any of the described the arrestin-1 variants are described. Nucleic acids encoding any of the described arrestin-1 variant can be made using techniques known in the art. In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GCNGCN (encoding E361A/D362A), GCNGGN (encoding E361A/D362G), GGNGCN (encoding E361G/D362A), GGNGGN (encoding E361G/D362G), GCNGAR (encoding E361A/D362), GGNGAR (encoding E361G/D362), GAYGCN (encoding E361/D362A), and GAYGGN (encoding E361/D362G), wherein N is A, G, C or T, Y is C or T, and R is A or G.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GCNGCN (encoding E365A/D366A), GCNGGN (encoding E365A/D366G), GGNGCN (encoding E365G/D366A), GGNGGN (encoding E365G/D366G), GCNGAR (encoding E365A/D366), GGNGAR (encoding E365G/D366), GAYGCN (encoding E365/D366A), and GAYGGN (encoding E365/D366G), wherein N is A, G, C or T, Y is C or T, and R is A or G.

In some embodiments, the nucleic acid encodes an arrestin-1 variant wherein the arrestin-1 variant has a glycine or alanine substitution at position 361 and/or 362 corresponding to SEQ ID NO: 30, has a reduced inhibitory effect on enolase-1 catalytic activity compared to wild-type arrestin-1, is able to bind to enolase-1, and comprises an amino acid sequence that is at least 70%, at least 75%, at least 80%, at least 85%, at least 90%, at least 95% identical to SEQ ID NO: 30.

In some embodiments, the nucleic acid encodes an arrestin-1 variant wherein the arrestin-1 variant has a glycine or alanine substitution at position 365 and/or 366 corresponding to SEQ ID NO: 17, has a reduced inhibitory effect on enolase-1 catalytic activity compared to wild-type arrestin-1, is able to bind to enolase-1, and comprises an amino acid sequence that is at least 70%, at least 75%, at least 80%, at least 85%, at least 90%, at least 95%, or 1005 identical to SEQ ID NO: 21-28 or an ortholog thereof.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GCNGCN (encoding E361A/D362A), wherein N is A, G, C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GCNGCN (encoding E365A/D366A), wherein N is A, G, C or T.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GCNGGN (encoding E361A/D362G), wherein N is A, G, C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GCNGGN (encoding E365A/D366G), wherein N is A, G, C or T.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GGNGCN (encoding E361G/D362A), wherein N is A, G, C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GGNGCN (encoding E365G/D366A), wherein N is A, G, C or T.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GGNGGN (encoding E361G/D362G), wherein N is A, G, C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GGNGGN (encoding E365G/D366G), wherein N is A, G, C or T.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GCNGAR (encoding E361A/D362), wherein N is A, G, C or T and R is A or G.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GCNGAR (encoding E365A/D366), wherein N is A, G, C or T and R is A or G.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GGNGAR (encoding E361G/D362), wherein N is A, G, C or T and R is A or G.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GGNGAR (encoding E365G/D366), wherein N is A, G, C or T and R is A or G.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GAYGCN (encoding E361/D362A), wherein N is A, G, C or T and Y is C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GAYGCN (encoding E365/D366A), wherein N is A, G, C or T and Y is C or T.

In some embodiments, the nucleic acid encoding an arrestin-1 variant comprises the sequence of SEQ ID NO: 29 or an ortholog thereof, wherein the sequence encoding the amino acids at positions 361 and 362 corresponding to SEQ ID NO: 30 is selected from the group consisting of: GAYGGN (encoding E361/D362G), wherein N is A, G, C or T and Y is C or T.

In some embodiments, the nucleic acid encoding an arrestin-1 variant comprises the sequence of SEQ ID NO: 17 or an ortholog thereof, wherein the sequence encoding the amino acids at positions 365 and 366 corresponding to SEQ ID NO: 18 is selected from the group consisting of: GAYGGN (encoding E365/D366G), wherein N is A, G, C or T and Y is C or T.

In some embodiments, the nucleic acid sequence encoding the arrestin-1 variant comprises the sequence of SEQ ID NO: 5, 7, 8, 19, or 20, or an ortholog thereof. In some embodiments, the nucleic acid encoding the arrestin-1 variant comprises a sequence that is at least 70%, at least 75%, at least 80%, at least 85%, at least 90%, or at least 95% identical to the sequence of SEQ ID NO: 5, 7, 8, 19, or 20.

### Vectors

Vectors containing nucleic acids encoding any of the described arrestin-1 variants can be made using methods known in the art for making or manufacturing such vectors. In some embodiments, the vectors are suitable for delivery of a nucleic acid to a mammalian tissue, such that the nucleic acid encoding the arrestin-1 variant is expressed. The tissue can be, but is not limited to, an eye. In some embodiments, the vector is suitable for subretinal injection, intravitreal injection, or suprachoroidal injection. In some embodiments, the vector is suitable for delivery to rod photoreceptor and/or cone photoreceptor cells. In some embodiments, the vector is suitable for delivery to rod photoreceptor and cone photoreceptor cells. In some embodiments, the vector is suitable for delivery to rod photoreceptor cells. In some embodiments, the vector is suitable for delivery to cone photoreceptor cells.

The vector can be, but is not limited to, a naked DNA, an mRNA, a plasmid, a non-viral nucleic acid vector, a CRISPR vector, or a viral vector. Non-viral nucleic acid vectors can be, but are not limited to: liposomes, lipoprotein particles, nanoparticles, polyplexes, lipid nanoparticles, polymeric delivery systems, and lipopolymers. The viral vector can be, but is not limited to, an AAV vector, an adenovirus, a retrovirus, a lentivirus, a vaccinia virus, an alphavirus, or a herpesvirus. An adeno-associated virus can be, but is not limited to, AAV1, AAV2, AAV2/1, AAV2/2, AAV2/4, AAV2/5, AAV2/6, AAV2/7, AAV2/8, AAV2/9, AAV4, AAV5, AAV6, AAV7, AAV7m8, AAV8, AAV9, and AAV44. An AAV2 can be, but is not limited to, an AAV2(quad Y-F+T-V) capsid serotype or an AAV2max-delHS. An AAV8 can be, but is not limited to, AAV8 Y733F. An AAV44 can be, but is not limited to, an AAV44.9(E5331D). For AAV vectors, the modified arrestin-1 gene can be packaged into the AAV using methods and constructs known in the art.

The vectors can be made using methods known in the art for making vectors suitable for use in gene therapy.

### In Vivo Administration

In pharmacology and toxicology, a route of administration is the path by which a drug, fluid, poison, or other substance is brought into contact with the body. In general, methods of administering drugs and nucleic acids for treatment of a mammal, including the eye, are well known in the art and can be applied to administration of the described compounds and compositions. The described compounds and compositions can be administered via any suitable route in a preparation appropriately tailored to that route. In general, any suitable method recognized in the art for delivering a therapeutic polypeptide or nucleic acid encoding the therapeutic polypeptide to the eye or retina can be adapted for use with the described arrestin-1 variants.

In some embodiments, the pharmaceutical compositions described herein can be formulated for administration to a subject. In some embodiments, the pharmaceutical compositions described herein can be formulated for administration to a retina of a subject. In some embodiments, the pharmaceutical compositions described herein can be formulated for administration to photoreceptors, rods, and/or cones, of a subject.

In some embodiments, a pharmaceutical composition described herein is administered by subretinal injection. In some embodiments, a pharmaceutical composition described herein is administered by intravitreal injection. In some embodiments, a pharmaceutical composition described herein is administered by suprachoroidal injection.

In some embodiments, a vector encoding an arrestin-1 variant is administered by subretinal injection. In some embodiments, a vector encoding an arrestin-1 variant is administered by intravitreal injection. In some embodiments, a vector encoding an arrestin-1 variant is administered by suprachoroidal injection.

### Pharmaceutical Composition

Any of the described arrestin-1 variants, nucleic acids encoding an arrestin-1 variant, or vectors containing a nucleic acid encoding an arrestin-1 variant can be provided in a pharmaceutical composition or medicament. A pharmaceutical composition or medicament comprising one or more arrestin-1 variants can be administered to a subject, such as a human or animal subject, for the treatment and/or prevention of symptoms and conditions associated with retinal degeneration.

A pharmaceutical composition or medicament includes a pharmacologically effective amount of at least one of the described arrestin-1 variants or nucleic acids encoding an arrestin-1 variant (including the described vectors) and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to (a) aid in processing of the drug delivery system during manufacture, (b) protect, support or enhance stability, bioavailability or patient acceptability of the API, (c) assist in product identification, and/or (d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents, and dispersing agents. A carrier may also contain isotonic agents, such as sugars, polyalcohols, sodium chloride, and the like into the compositions.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.). It is also envisaged that cells, tissues, or isolated organs that express or comprise the one or more of the described agents may be used as “pharmaceutical compositions”.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

### Methods of Treatment

Disclosed herein are methods of treatment or retinal degeneration or prevention of conditions or symptoms associated with a retinal degeneration or a retinal degenerative disease, the methods comprising administering a therapeutically effective amount of one or more of the described arrestin-1 variants to the retina of a subject. A retinal degenerative disease can be a disease associated with loss of rod and/or cone photoreceptors. Diseases and/or disorders that would benefit from administration of an arrestin-1 variant include, but are not limited to retinitis pigmentosa, cone-rod dystrophies, or Usher's syndromes. In some embodiments, the methods comprise administering to the eye of the subject the arrestin-1 variant. Administering one or more of the described arrestin-1 variants includes administering one or more arrestin-1 variant polypeptides, one or more nucleic acids encoding one or more arrestin-1 variants, one or more vectors containing one or more nucleic acids encoding one or more arrestin-1 variants, or one or more pharmaceutical compositions containing an arrestin-1 variant or nucleic acid encoding an arrestin-1 variant.

In some embodiments, administration of an arrestin-1 variant can be used to decrease the number, severity, and/or frequency of symptoms of a retinal degeneration in a subject.

Administering an arrestin-1 variant can be used to treat at least one symptom of retinal degeneration in a subject having a retinal degenerative disease. The subject is administered a therapeutically effective amount of one or more arrestin-1 variants, thereby treating the at least one symptom.

In some embodiments, the described arrestin-1 variants are useful for treating, preventing, or managing clinical presentations associated with a retinal degenerative disease. The methods comprise administering to a subject in need of such treatment, prevention, or management a therapeutically or prophylactically effective amount of one or more of the described arrestin-1 variants. In some embodiments, administration of an arrestin-1 variant can be used to decrease the number, severity, and/or frequency of symptoms of retinal degeneration in a subject.

In some embodiments, one or more arrestin-1 variants can be used to treat a subject at risk of developing a retinal degenerative disease. The described arrestin-1 variants and methods of using the arrestin-1 variants can be used to prevent or delay onset of at least one symptom associated with retinal degeneration in a subject having a retinal degenerative disease or at risk of developing a retinal degenerative disease. In some embodiments, the subject is administered a prophylactically effective amount of any one or more of the described arrestin-1 variants thereby preventing or delaying onset of the at least one symptom.

The described arrestin-1 variants may be delivered for research purposes or to produce a change in a retina, photoreceptor, rod, or cone that is therapeutic.

Conditions or symptoms associated with retinal degenerative disease include, but are not limited to, degradation of photoreceptors, retinal degeneration, visional loss, loss of scotopic vision, loss of photopic vision, and loss or photoreceptors

An arrestin-1 variant can be administered to the retina of a subject to: increase survival of photoreceptors, improve photoreceptor function, protect photoreceptors against degeneration, decreases degeneration of rod cell and/or the cone cells, increases the rate of glycolysis in rod cells and/or cone cells, improve or preserve scotopic and/or photopic vision and/or delay onset of photoreceptor degeneration.

In some embodiments, administration of an arrestin-1 variant to a retina increase glycolysis in the retina. Increase glycolysis in the retina can be measured by determining lactate production or lactate production rate. In some embodiments, administering an arrestin-1 variant to a retina increase lactate production rate by at least 5%, at least 10%, at least 15%, at least 20%, or at least 24%.

In some embodiments, the subject is a mammal, including, but not limited to, a human patient. In some embodiments, the method comprises administering a composition comprising an arrestin-1 variant described herein to a mammal to be treated.

### EXAMPLES

### Example 1. General Methods

A. Mutagenesis. Cysteine substitution mutations and charge reversal mutations were introduced into N-terminally His(6)-tagged bovine arrestin-1 by overlapping PCR amplification of the bovine arrestin-1 cDNA as previously described (McDowell et al. (1999) Sulfhydryl reactivity demonstrates different conformational states for arrestin, arrestin activated by a synthetic phosphopeptide, and constitutively active arrestin. Biochem 38, 6119-6125). For the cysteine substitution mutants, the arrestin-1 cDNA template also contained the two cysteine mutations, C63A and C143A, to remove the two reactive cysteines that are endogenously present in arrestin-1. Arrestin-1 containing combinations of mutants was prepared by serial mutagenesis, using the mutagenized cDNA as the template to add the next mutation, until all desired mutations were introduced. The primers used to introduce these cysteine substitutions and charge reversal mutations are shown in Table 1. The altered cDNA's were cloned into pPICZ-A at the EcoRI site. Amino acid substitutions at other sites were made similarly.

TABLE 1
Targeted mutations and primer pairs used for site-directed mutagenesis in
arrestin-1^{a}.

| Targeted | Synthetic overlapping oligonucleotide pairs for mutagenesis | SEQ ID |
|---|---|---|
| Substitution^{b} | (5′→-3′) | NO. |
| H10C | AAGCCCGCACCAAACTGTGTTATCTTCAAGAAG | 31 |
| | CTTCTTGAAGATAACACAGTTTGGTGCGGGCTT | 32 |
| R18C | TTCAAGAAGATCTCCTGTGATAAATCGGTGACC | 33 |
| | GGTCACCGATTTATCACAGGAGATCTTCTTGAA | 34 |
| Y25C | GATAAATCGGTGACCATCTGTCTGGGGAAGAGAGATTAC | 35 |
| | GTAATCTCTCTTCCCCAGACAGATGGTCACCGATTTATC | 36 |
| K28C | GACCATCTACCTGGGGTGTAGAGATTACATAGAC | 37 |
| | GTCTATGTAATCTCTACACCCCAGGTAGATGGTC | 38 |
| R37C | ATAGACCACGTTGAATGTGTAGAGCCTGTGGATG | 39 |
| | CATCCACAGGCTCTACACATTCAACGTGGTCTAT | 40 |
| E50C | GTGCTGGTGGATCCATGTCTCGTGAAGGGCAAG | 41 |
| | CTTGCCCTTCACGAGACATGGATCCACCAGCAC | 42 |
| K53C | GATCCTGAGCTCGTGTGTGGCAAGAGAGTGTAC | 43 |
| | GTACACTCTCTTGCCACACACGAGCTCAGGATC | 44 |
| I72C | TACGGCCAGGAAGACTGTGACGTGATGGGC | 45 |
| | GCCCATCACGTCACAGTCTTCCTGGCCGTA | 46 |
| S86C | CAGGGACCTCTACTTCTGTCAAGTCCAGGTGTTCC | 47 |
| | GGAACACCTGGACTTGACAGAAGTAGAGGTCCCTG | 48 |
| V94C | CAGGTGTTCCCTCCATGTGGGGCCTCGGGCGCC | 49 |
| | GGCGCCCGAGGCCCCACATGGAGGGAACACCTG | 50 |
| A113C | ATCAAGAAGCTGGGCTGTAACACCTACCCCTTC | 51 |
| | GAAGGGGTAGGTGTTACAGCCCAGCTTCTTGAT | 52 |
| Y125C | CTCACGTTTCCTGACTGTTTGCCCTGTTCGGTG | 53 |
| | CACCGAACAGGGCAAACAGTCAGGAAACGTGAG | 54 |
| V139C | CCAGCTCCGCAAGATTGTGGCAAGAGCGCAGGG | 55 |
| | CCCTGCGCTCTTGCCACAATCTTGCGGAGCTGG | 56 |
| K166C | GAGGACAAAATTCCCTGTAAGAGCTCCGTGCG | 57 |
| | CGCACGGAGCTCTTACAGGGAATTTTGTCCTC | 58 |
| D183C | CAGCACGCGCCACGATGTATGGGTCCCCAGCCCCG | 59 |
| | CGGGGCTGGGGACCCATACATCGTGGCGCGTGCTG | 60 |
| R189C | GATATGGGTCCCCAGCCCTGTGCCGAGGCCTCCTGG | 61 |
| | CCAGGAGGCCTCGGCACAGGGCTGGGGACCCATATC | 62 |
| W194C | CGAGCCGAGGCCTCCTGTCAGTTCTTCATGTCG | 63 |
| | CGACATGAAGAACTGACAGGAGGCCTCGGCTCG | 64 |
| S199C | TGGCAGTTCTTCATGTGTGACAAGCCCCTGCGC | 65 |
| | GCGCAGGGGCTTGTCACACATGAAGAACTGCCA | 66 |
| S210C | CTCGCCGTCTCGCTCTGTAAAGAGATCTATTAC | 67 |
| | GTAATAGATCTCTTTACAGAGCGAGACGGCGAG | 68 |
| E218C | ATCTATTACCACGGGTGTCCCATTCCTGTGACC | 69 |
| | GGTCACAGGAATGGGACACCCGTGGTAATAGAT | 70 |
| S231C | GTGACCAACAGCACATGTAAGACAGTGAAGAAG | 71 |
| | CTTCTTCACTGTCTTACATGTGCTGTTGGTCAC | 72 |
| S251C | CGTGGTTCTCTACTGTAGTGATTATTAC | 73 |
| | GTAATAATCACTACAGTAGAGAACCACG | 74 |
| K267C | GAGGAAGCACAGGAATGTGTGCCGCCAAACAGC | 75 |
| | GCTGTTTGGCGGCACACATTCCTGTGCTTCCTC | 76 |
| V281C | AAGACGCTGACGCTGTGTCCCTTGCTGGCCAAC | 77 |
| | GTTGGCCAGCAAGGGACACAGCGTCAGCGTCTT | 78 |
| E302C | GGGAAAATCAAGCACTGTGACACGAACCTGGCC | 79 |
| | GGCCAGGTTCGTGTCACAGTGCTTGATTTTCCC | 80 |
| D317C | CATAAAGGAGGGAATATGTAAGACCGTCATGGGG | 81 |
| | CCCCATGACGGTCTTACATATTCCCTCCTTTATG | 82 |
| D362C | CATCCCCAGCCAGAGTGTCCAGATACCGCCAAG | 83 |
| | CTTGGCGGTATCTGGACACTCTGGCTGGGGATG | 84 |
| E393C | GCAGGAGAATATAAGTGTGAGAAGACAGACCAG | 85 |
| | CTGGTCTGTCTTCTCACACTTATATTCTCCTGC | 86 |
| R29E | CATCTACCTGGGGAAGGAAGATTACATAGACCAC | 87 |
| | GTGGTCTATGTAATCTTCCTTCCCCAGGTAGATG | 88 |
| E36K | GATTACATAGACCACGTTAAACGAGTAGAGCCTGTG | 89 |
| | CACAGGCTCTACTCGTTTAACGTGGTCTATGTAATC | 90 |
| R37D | CATAGACCACGTTGAAGATGTAGAGCCTGTGGATG | 91 |
| | CATCCACAGGCTCTACATCTTCAACGTGGTCTATG | 92 |
| D183K | CAGCACGCGCCACGCAAGATGGGTCCCCAGCC | 93 |
| | GGCTGGGGACCCATCTTGCGTGGCGCGTGCTG | 94 |
| E302K | GGGAAAATCAAGCACAAGGACACGAACCTGGCC | 95 |
| | GGCCAGGTTCGTGTCCTTGTGCTTGATTTTCCC | 96 |
| E361K | CATGCATCCCCAGCCAAAGGACCCAGATACCGCC | 97 |
| | GGCGGTATCTGGGTCCTTTGGCTGGGGATGCATG | 98 |
| D362K | CATCCCCAGCCAGAGAAGCCAGATACCGCCAAG | 99 |
| | CTTGGCGGTATCTGGCTTCTCTGGCTGGGGATG | 100 |
| E361G/D362G | CATGCATCCCCAGCCAGGAGGTCCAGATACCGCCAAGG | 101 |
| | CCTTGGCGGTATCTGGACCTCCTGGCTGGGGATGCATG | 102 |

^{a}The arrestin-1 cDNA utilized contained an N-terminal His(6) tag introduced after the initiating methionine and with C63A and Cl43A mutations to remove the two reactive cysteines.
^{b}Numbering corresponds to bovine arrestin, SEQ ID NO: 30

B. Protein production. Plasmids encoding the arrestin-1 variants were heterologously expressed in Pichia pastoris for three days in the presence of 0.5% methanol. Following disruption of the cell wall by French pressing (20,000 psi), the arrestin-1 protein was purified to >95% homogeneity by chromatographic purification over nickel-agarose affinity resin (GE resin) in 50 mM sodium phosphate (pH 8.0) with 300 mM sodium chloride and 10 mM imidazole, eluting with 100 mM ethylenediaminetetraacetate. Fractions containing purified arrestin-1 protein were pooled, and dialyzed against LAP200^{N} buffer (50 mM HEPES, 200 mM NaCl, 1 mM EGTA, 1 mM MgCl₂, 10% glycerol, 0.05% NP-40, pH 7.4).

C. Quenching assay. Cysteine mutants of arrestin-1 were labeled with monobromobimane (mBBr, ThermoFisher), reacting the arrestin-1 protein with 100-fold molar excess of the fluorescent label for two hours at room temperature. The unreacted label was removed by dialyzing against three sequential changes of 500-volumes of LAP200 buffer. Fluorescence labeling of the arrestin-1 was quantified by measuring the absorbance of mBBr (394 nm, λmax) and arrestin-1 (278 nm, λmax) and calculating the percent labeled (assuming E_{MBB, 394 nm}=5,300 M⁻¹ cm⁻¹ and E_{Arr1, 278 nm}=25,200 M⁻¹ cm⁻¹). Fluorescence quenching of the mBBr fluorophore was performed using 0.25 μM mBBr-labeled arrestin-1 with 100 mM potassium iodide in the presence or absence of 5 μM bovine enolase-1 that had been heterologously expressed and purified as previously described for arrestin-1, monitoring fluorescence emission at 470 nm. To determine the degree to which enolase-1 shielded a residue from quenching by potassium iodide, a protection factor was calculated as follows:

$${P}_{f} = \frac{{F}_{( A + E + K )} - {F}_{( A + K )}}{{F}_{( A )} - {F}_{( A + K )}}$$

where F_{(A)} is the fluorescence of mBBr-labeled arrestin-1 in solution alone, F_{(A+K)} is the fluorescence of mBBr-arrestin-1 in the presence of potassium iodide, and F_{(A+E+K)} is the fluorescence of mBBr-arrestin-1 in the presence of both enolase-1 and potassium iodide. The protection quotient looks at the total range of protection offered by enolase-1 normalized to the access of the labeled cysteine to the potassium iodide. Values near zero indicated no protection of the fluorophore-labeled cysteine. The protection factor will approach one if enolase-1 completely shields the mBBr from quenching by the potassium iodide.

D. Molecular Modeling. The interaction of arrestin-1 and enolase-1 was modeled using the crystallographic structure of arrestin-1 (1CF1, Chain A, (Hirsch et al. (1999) The 2.8 angstrom crystal structure of visual arrestin: A model for arrestin's regulation. Cell 97, 257-269) and enolase-1 (3B97, (Kang et al. (2008) Structure of human alpha-enolase (hENO1), a multifunctional glycolytic enzyme. Acta Crystallogr D Biol Crystallogr 64, 651-657), using ClusPro 2.0 (Kozakov et al. (2013) How good is automated protein docking? Proteins: Structure, Function, and Bioinformatics 81, 2159-2166, Kozakov et al. (2017) The ClusPro web server for protein-protein docking. Nature Protoc 12, 255-278, and Vajda et al. (2017) New additions to the ClusPro server motivated by CAPRI. Proteins 85, 435-444). Initial models were generated with no constraints, using arrestin-1 as the “receptor” molecule and enolase-1 as the “ligand”. The model was refined by constraining interaction sites to include residues that showed the strongest protection of fluorescence quenching by enolase-1, namely His-10, Asp-183, Glu-218, Glu-302, and Asp-362.

E. Immunoprecipitation/Enolase Pulldown assay. The interaction between arrestin-1 and enolase-1 was assessed by using anti-arrestin-1 antibody to immunoprecipitate the arrestin-1-enolase-1 complex in which the enolase-1 was fluorescently labeled with AlexaFluor546. For this assay, 1.5 mg of Protein G-coated magnetic beads (DynaBeads, ThermoFisher), were coated with 10 μg of purified C10C10 anti-arrestin-1 monoclonal antibody (Donoso et al. (1990) S-antigen: preparation and characterization of site-specific monoclonal antibodies. Curr Eye Res 9, 343-355). Enolase-1 was labeled on its reactive cysteines with AlexaFluor-546 maleimide (ThermoFisher), reacting the enolase-1 with 100-fold molar excess of the fluorescent label. The fluorescently labeled enolase-1 was then sequentially dialyzed in LAP200^{N} buffer to remove any unreacted label. For the immunoprecipitation of the arrestin-1-enolase-1 complex, 5 μM arrestin-1 (or arrestin-1 variant) was mixed with 5 μM enolase-1-Alexa546 in LAP200^{N} in a 200 μL final volume for 2 h at 4° C., to which 10 μg of anti-arrestin-1 antibody on magnetic beads was subsequently added for 16 h with gentle rotation. The beads were magnetically captured, washed ten times with LAP200^{N} buffer, and then eluted with 0.1 M glycine (pH 2.5). After neutralizing the pH with 0.1 volume 1.5 M Tris base (pH 8.5), the fluorescence of the captured enolase-1-Alexa546 was measured, exciting fluorescence at 530 nm, and measuring average emission at 570-575 nm with a dual-detector fluorimeter (QM-1 steady state fluorescence spectrophotometer, Photon Technologies Inc).

F. Enolase activity assay. The catalytic activity of enolase-1 was measured by monitoring ATP production from the processing of phosphoenolpyruvate produced by enolase catalysis of 2-phosphoglycerate. For the reaction, 2 mM 2-phospho-glycerate (Sigma), 2 mM adenosine diphosphate (Sigma), and 0.3 U/mL pyruvate kinase (MP Biochemicals) were mixed with 50 nM enolase-1 with 0-1.6 μM arrestin-1 or arrestin-1 variant. ATP production was monitored using a luciferase luminescence assay, mixing equal volumes of the reaction mixture and firefly luciferase (CellTiter-Glo 2.0 Cell Viability Assay, Promega). Luminescence was monitored at 550-570 nm at 5 min intervals for 40 minutes, calculating the rate of luminescence production from a linear regression of luminescence as a measure of enolase catalytic activity.

### Example 2. Fluorescence Mapping of Interaction Between Arresting-1 and Enolase-1

Arrestin-1 selectively interacts with enolase-1 in photoreceptors, modulating the catalytic activity of enolase-1 (Smith et al. (2011) Interaction of arrestin with enolase1 in photoreceptors. Invest Ophthalmol Vis Sci 52, 1832-1840). We investigated the biophysical nature of the interaction between arrestin-1 and enolase-1 and the role of arrestin-1 in modulating catalytic activity of enolase-1. Targeted fluorescence labeling of arrestin-1 and fluorescence quenching was used to map the surface of arrestin-1 that interacts with enolase-1. 28 cysteine substitutions were introduced individually into arrestin-1 at residues that are positioned across the surface of arrestin-1. Locations of the cysteine substitutions are shown in Table 2. These cysteine substitutions were introduced into an arrestin-1 in which Cys-63 and Cys-143 were converted to alanine to remove the endogenous reactive cysteines in arrestin-1. These introduced cysteine residues were labeled with the thiol-reactive fluorophore monobromobimane (mBBr).

TABLE 2
Fluorescence quenching of mBBr-labeled arrestin-1 cysteine
mutants by potassium iodide with protection by enolase-1.

| Cysteine | | | | |
|---|---|---|---|---|
| Substitution | F_{(A)} | F_{(A+K)} | F_{(A+E+K)} | P_{f} |
| H10C | 12595.7 | 1449.61 | 10279.5 | 0.792 |
| R18C | 40060.9 | 1148.8 | 963.2 | −0.005 |
| Y25C | 17932.1 | 789.91 | 599.8 | −0.011 |
| K28C | 23275.8 | 557.8 | 17195.5 | 0.732 |
| R37C | 28962.6 | 682.3 | 16642.6 | 0.564 |
| E50C | 25319.4 | 891.0 | 8143.2 | 0.297 |
| K53C | 34263.2 | 421.3 | 11031.5 | 0.314 |
| I72C | 40304.8 | 549.0 | 630.0 | 0.002 |
| S86C | 20069.2 | 5060.5 | 6159.6 | 0.073 |
| V94C | 16472.6 | 935.5 | 1901.7 | 0.062 |
| A113C | 25323.9 | 750.9 | 3465.0 | 0.109 |
| Y125C | 44782.9 | 13617.9 | 13978.0 | 0.012 |
| V139C | 27086.9 | 813.6 | 778.3 | −0.001 |
| K166C | 24572.6 | 404.6 | 1041.5 | 0.026 |
| D183C | 24307.5 | 538.9 | 19234.9 | 0.787 |
| R189C | 23296.8 | 490.8 | 11133.8 | 0.467 |
| W194C | 44782.9 | 13617.9 | 13978.0 | 0.012 |
| S199C | 23297.2 | 662.5 | 636.1 | −0.001 |
| S210C | 13660.6 | 724.1 | 8717.0 | 0.618 |
| E218C | 8846.4 | 953.6 | 9498.0 | 1.083 |
| S231C | 6973.8 | 682.0 | 694.6 | 0.002 |
| S251C | 6123.7 | 674.5 | 730.5 | 0.011 |
| K267C | 5523.2 | 350.3 | 2851.8 | 0.484 |
| V281C | 6002.9 | 1167.2 | 2439.3 | 0.269 |
| E302C | 4562.3 | 686.6 | 3676.4 | 0.772 |
| D317C | 33509.8 | 11518.1 | 11514.0 | 0.002 |
| D362C | 4735.3 | 950.7 | 5491.7 | 1.200 |
| E393C | 3604.6 | 542.6 | 952.8 | 0.134 |

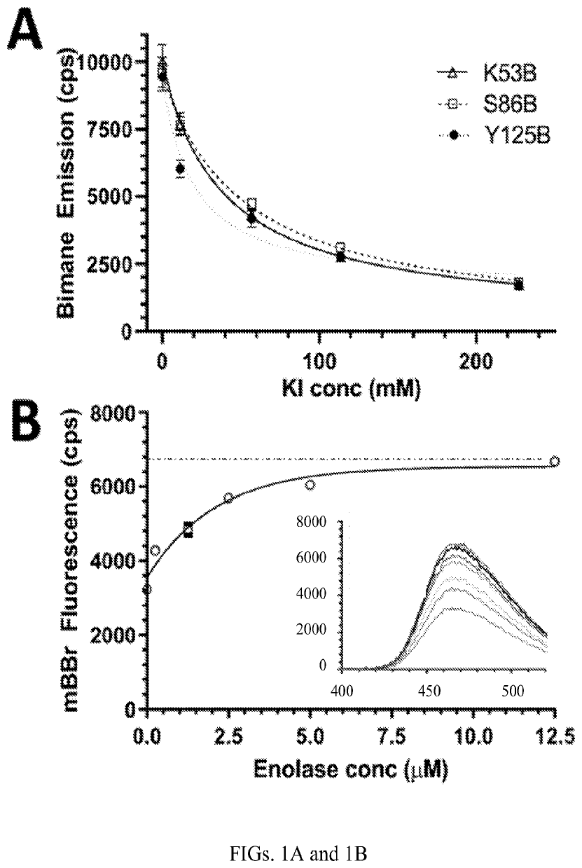

A. Analysis of quenching of the fluorophore by potassium iodide (KI). KI concentration for use in the quenching experiments was determined by performing a titration assay using three different cysteine-substituted arrestin-1 variants, K53C, S86C, and Y125C, each labeled with monobromobimane (mBBr) and quenching with increasing concentrations of KI (FIG. 1A). For all three labeled positions, 50% quench of fluorescence of the 3 μM arrestin-bimane was observed at ˜33 mM KI, or with a 10⁴ molar excess of KI over the bimane-labeled arrestin.

B. Enolase concentration. The concentration of enolase-1 that could provide protection of mBBr fluorescence from quenching by the KI was then titrated. 0.25 μM H10C-arrestin-1 labeled with mBBr was incubated with increasing concentrations of enolase-1 prior to the addition of 2.5 mM KI, and fluorescence emission of the mBBr was measured (FIG. 1B). A clear concentration dependence for the effect of enolase on the quenching was observed, with 12.5 μM enolase-1 providing protection of the fluorophore equivalent to the fluorescence in the absence of any KI (black trace, inset). 90% quench protection was selected as the criterion value, which corresponded to a 20-fold molar excess of enolase-1 to arrestin-1.

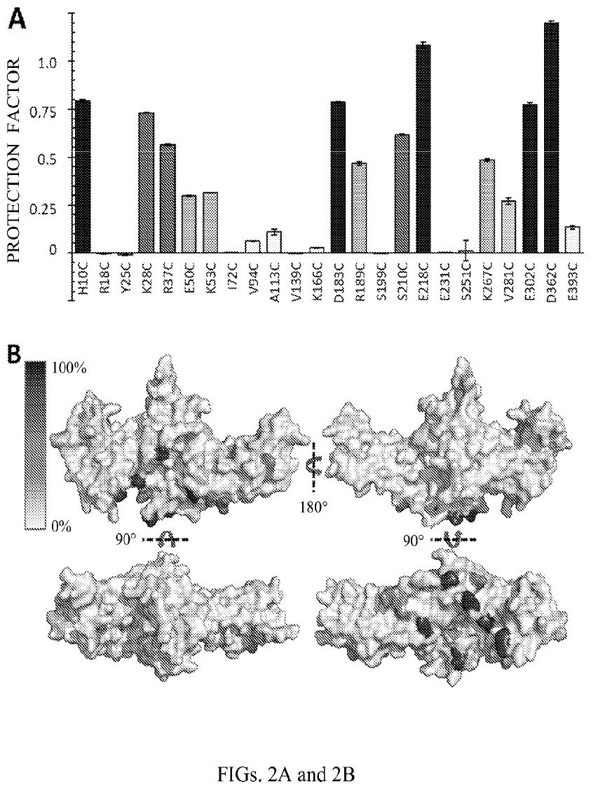

C. Mapping interaction of arrestin-1 with enolase-1. Using these parameters of ˜50% quench with 10⁴ molar excess of KI over arrestin-1 and 20-fold molar excess of enolase-1 over arrestin-1, the arrestin-1 cysteine point mutations were analyzed for the potential of enolase-1 to protect the mBBr-labeled arrestin-1 for fluorescence quenching. The fluorescence emission of the mBBr-labeled arrestin-1 with and without potassium iodide was measured to determine the range of quenching by KI. The fluorescence emission quench by KI with and without enolase-1 was then measured. A quench protection factor was calculated as described in the experimental procedures (Table 2). Quench protection analysis revealed a range of residues that when labeled by mBBr were highly protected from quenching by enolase (FIG. 2A). When mapped onto the surface of the arrestin-1 structure, these residues largely mapped to a single surface along the “bottom” of the arrestin protein (FIG. 2B).

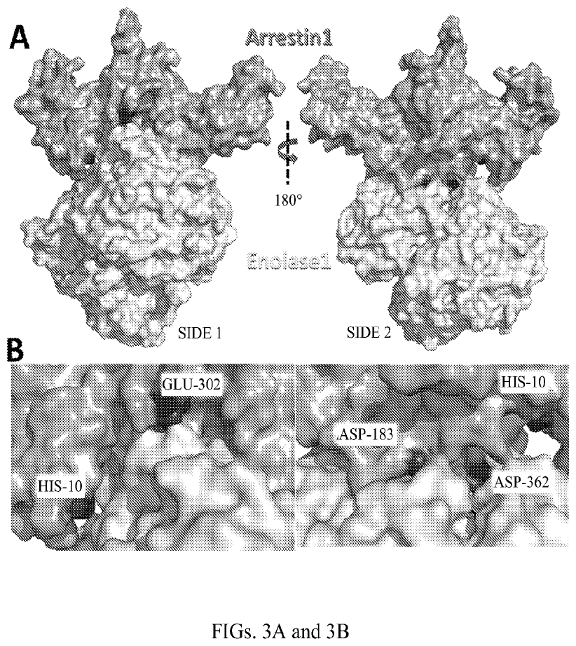

A molecular model of the interaction between arrestin-1 and enolase-1 was generated by performing energy minimization docking of arrestin-1 with an enolase-1 dimer using ClusPro 2.0. A dimer of enolase-1 was used for this model since this is the typical physiological state of enolase-1 (Wold (1971) Enolase in The enzymes, 3rd edn (Boyer, P. D. ed.), Academic Press, New York. pp 499-538), and previous data showed that arrestin-1 crosslinks to a dimer of enolase-1 (Smith et al. (2011) Interaction of arrestin with enolase1 in photoreceptors. Invest Ophthalmol Vis Sci 52, 1832-1840). Based on the fluorescence quench studies above, the interaction sites were constrained to include residues His-10, Asp-183, Glu-218, Glu-302, and Asp-362 of arrestin-1. The resulting model predicted both N- and C-terminal domains of arrestin-1 to bind to a single unit of the enolase-1 dimer (FIGS. 3A and 3B), forming numerous interactions with surface residues, including several paired charges interactions.

Mapping of the arrestin-1/enolase-1 interaction through targeted fluorophore quenching showed that labeling of the arrestin-1 molecule was best protected when the fluorophore was positioned at residues along a single side of the arrestin-1 molecule. This enolase-1-interacting surface was opposite the side that has been shown to engage its G protein-coupled receptor, photo-activated rhodopsin (Kang et al. (2016) A structural snapshot of the rhodopsin-arrestin complex. FEBS J 283, 816-821; Kang et al. (2015) Crystal structure of rhodopsin bound to arrestin by femtosecond X-ray laser. Nature 523, 561-567; and Zhou et al. (2016) X-ray laser diffraction for structure determination of the rhodopsin-arrestin complex. Sci Data 3, 160021). This finding is consistent with our observation that the binding of arrestin-1 to light-activated, phosphorylated rhodopsin did not exclude the binding of enolase-1.

This model for how arrestin-1 impacts enolase-1 activity also offers a potential explanation for the previous observation that arrestin-1 has a maximum effect of reducing enolase-1 activity by only ˜25% (Smith et al. (2011) Interaction of arrestin with enolase1 in photoreceptors. Invest Ophthalmol Vis Sci 52, 1832-1840). Interaction of arrestin-1 with only one of the active sites in the enolase-1 dimer, the maximum effect that would be expected is only a 50% reduction in enolase activity, even if arrestin-1 completely disrupted the active site

### Example 3. Mutational Mapping of Interaction Between Arresting-1 and Enolase-1

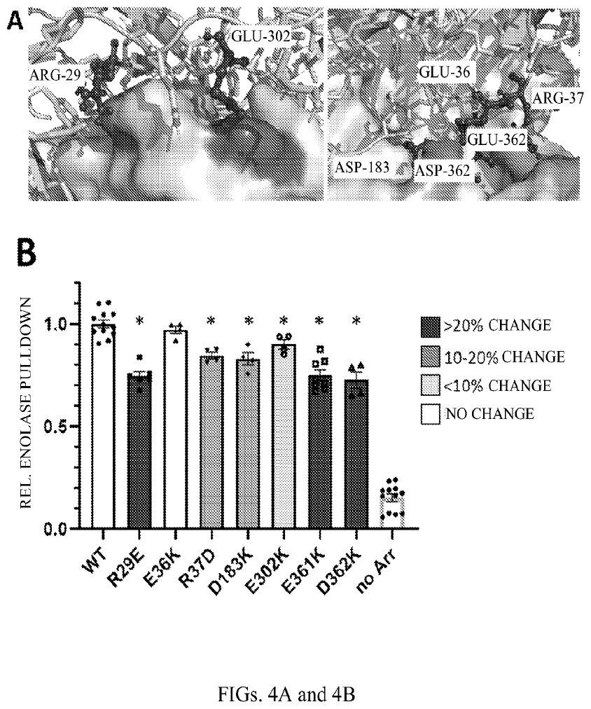

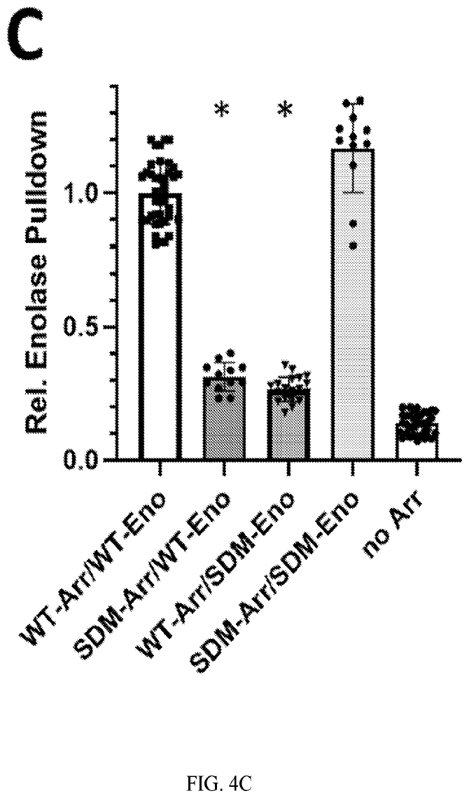

The charge-pair interactions identified in example 1 were selected to further investigate the modeled arrestin-1/enolase-1 interaction. Seven charged-pair interactions (Table 3 and FIG. 4A) were identified from the model as having the closest interactions. These seven residues were singly mutated on the arrestin-1 protein to reverse the charge of the amino acid side chain, while approximately preserving side chain size (e.g., Glu to Lys, or Arg to Asp). Following heterologous expression and purification, each arrestin-1 mutant was then assessed for its interaction with enolase-1, using an anti-arrestin-1 immunoprecipitation assay to pulldown fluorescently labelled enolase-1. In this assay, mutations R29E, E361K, and D362K reduced the pulldown of enolase-1 by ˜25% (FIG. 4B). Mutations R37D, D183K, and E302K had a smaller, but also significant effect on interaction with enolase-1. The significant effect of each of these amino acid substitutions, with the exception of E36K, on the interaction with enolase-1 suggests that the model of interaction between arrestin-1 and enolase-1 (FIGS. 3A and 3B) is correct.

TABLE 3
Charged-pair interactions between arrestin-1 and enolase-1
in the energy minimized docking model shown in FIGS.
3A and 3B. The amino acid change made for charge reversal
in the various arrestin-1 and enolase-1 mutants are
indicated in parentheses after each amino acid.

| Arr1 residue | Enolase-1 residue | Molecular |
|---|---|---|
| (charge reversal) | (charge reversal) | Distance (Å) |
| Arg-29 (Glu) | Asp-265 (Lys) | 2.8 |
| Glu-36 (Lys) | Lys-53 (Glu) | 2.7 |
| Arg-37 (Asp) | Glu-197 (Lys) | 1.9 |
| Asp-183 (Lys) | Lys-59 (Asp) | 2.3 |
| Glu-302 (Lys) | Lys-255 (Asp) | 1.8 |
| Glu-361 (Lys) | Lys-196 (Glu) | 2.6 |
| Asp-362 (Lys) | Lys-192 (Glu) | 2.5 |

Charge reversal of the amino acid side chain on arrestin-1 for six out of the seven identified pairs had a significant impact on the binding of enolase-1 to the modified arrestin-1 (FIG. 4B, Table 3). Significantly, combining all seven charge reversals into a single protein resulted in an arrestin-1 with no measurable binding to enolase-1 in our immunoprecipitation assay. Assembly of all seven point mutations (Table 3) into a single arrestin-1 protein was generated (SDM-Arr). The corresponding charge reversals (Table 3) was also assembled in enolase-1 (SDM-Eno). Arrestin-1 containing all seven charge reversal substitutions decreased pulldown of enolase by over 80% compared to the wild-type arrestin-1 (FIG. 4C). Similarly, pulldown of enolase-1 containing all seven charge reversal substitutions by wild-type arrestin-1 was decrease by over 80% compared to pulldown of wild-type enolase-1. When SDM-Arr was paired with the SDM-Eno, binding interaction was restored (FIG. 4C), indicated these amino acids on arrestin-1 and enolase-1 form complementary pairs of interactions.

### Example 4. Effect of Arrestin-I Variants on Enolase-1 Binding and Enolase-1 Catalytic Activity

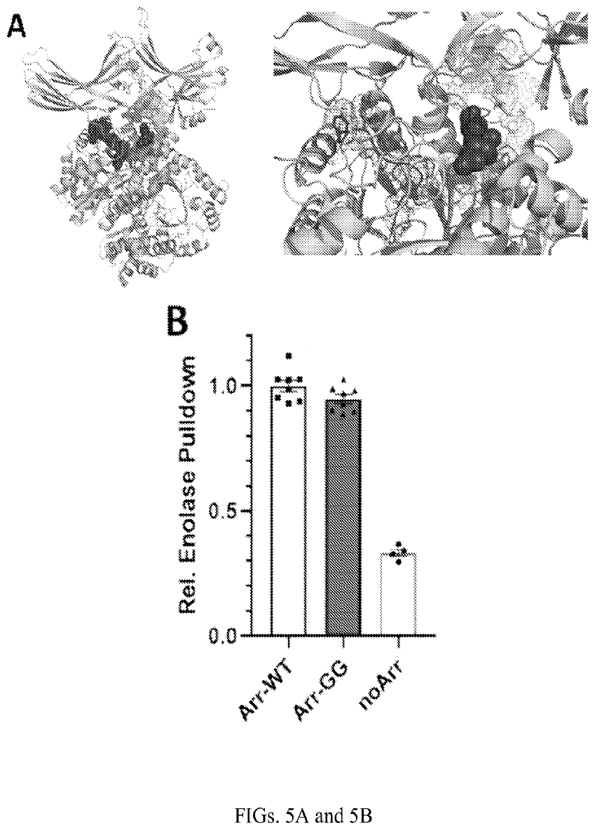

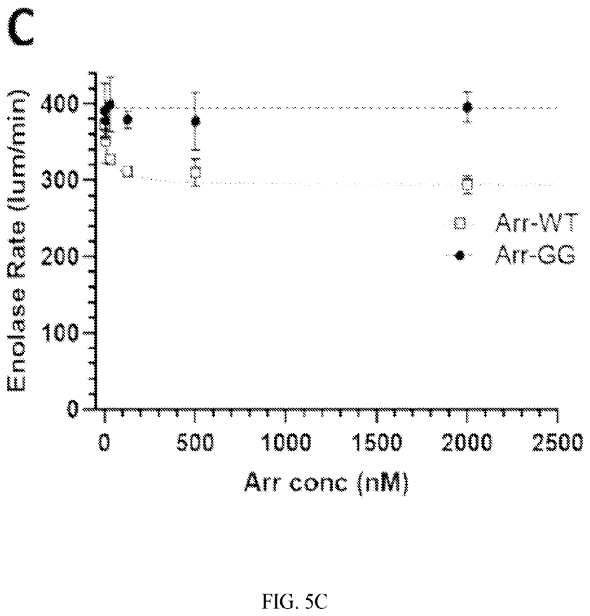

It has previously been shown that interaction of arrestin-1 with enolase-1 decreases catalytic activity enolase-1 by ˜25%. Enolase-1 loops L1, L2, and L3, and the magnesium coordinating residues Ser-36, Asp-244, Glu-292, and Asp-317 comprise the key components of the enolase-1 active site (Kang et al. (2008) Structure of human alpha-enolase (hENO1), a multifunctional glycolytic enzyme. Acta Crystallogr D Biol Crystallogr 64, 651-657 and Schreier et al. (2010) Engineering the enolase magnesium II binding site: Implications for its evolution. Biochem 49, 7582-7589). Using the model generated in examples 2 and 3, Glu-361 and Asp-362 were identified as residues that insert into enolase-1. Further, these two residues were identified as being in close proximity to all three of the loops that comprise one of the active sites in the enolase-1 dimer, particularly Ser-156 and Gly-159 in loop L2 (FIG. 5A). The proximity of these residues to loops L1, L2, and L3 was 7.6 Å, 4.9 Å, and 6.0 Å respectively. Glu-361 and Asp-362 to mutated to glycine to analyze the effect of reducing the size and/or charge of the side chains of these amino acids on the inhibitory effect of arrestin-1 on enolase-1.

The effects of the E361G and D362G substitutions of enolase-1 binding were analyzed using the pulldown assay described above. The pulldown of enolase-1 with the E361G/D362G arrestin-1 variant was indistinguishable from that of wild-type arrestin-1 (FIG. 5B). Thus, mutation of these two amino acids of arrestin-1 do not significantly disrupt the overall interaction between arrestin-1 and enolase-1.

The effects of the E361G and D362G substitutions of enolase-1 catalytic activity was then analyzed (Table 4). The catalytic activity of enolase was measured by monitoring the production of ATP from the processing of 2-phosphoglycerate to pyruvate (FIG. 5C, inset). As predicted, wild-type arrestin-1 inhibited the catalytic activity of enolase-1 by approximately 25%. Significantly, this inhibition of enolase catalysis was completely absent in the E361G/D362G arrestin-1 variant (FIG. 5C), even at 320-fold molar excess. Selective mutation of E361 and/or D362 on arrestin-1 can reduce its inhibitory effect on enolase-1 catalytic activity without substantially disrupting binding interaction with enolase-1. The E361G/D362G arrestin-1 variant maintains the ability to bind to enolase-1 without adversely affecting enolase-1 catalytic activity. Without wishing to be bound by theory, it is possible that reducing the size and/or charge of the amino acids at positions 361 and 362 of arrestin-1 reduces steric and/or charge interference in positioning of 2-phosphoglycerate in the active site of enolase-1 when arrestin-1 is bound to enolase-1.

TABLE 4
Inhibition of enolase-1 catalytic activity by arrestin-1 variants.

| Arrestin-1 | % inhibition |
|---|---|
| Wild-type | 25.2 |
| E361G | 18.3 |
| E361A | 21.1 |
| D362G | 20.7 |
| D362A | 22.2 |
| E361A/D362A | 9.0 |
| E361A/D362G | 1.3 |
| E361G/D362A | 8.3 |
| E361G/D362G | 0.7 |

Photoreceptors are highly polarized and dynamic in nature. As a modified sensory cilium, the inner segment portion of rods and cones is responsible for most of the metabolic activity of the photoreceptor, including glycolysis, whereas the outer segment is principally responsible for phototransduction. Accordingly, enolase-1 principally localizes to the inner segment of photoreceptors along with the other glycolytic enzymes (Smith et al. (2011) Interaction of arrestin with enolase1 in photoreceptors. Invest Ophthalmol Vis Sci 52, 1832-1840 and Hsu et al. (1991) Glycolytic enzymes and a GLUT-1 glucose transporter in the outer segments of rod and cone photoreceptor cells. J Biol Chem 266, 21745-21752). In contrast, arrestin-1 localization is dynamic, translocating from the inner segment under dark conditions to the outer segment upon light exposure (Broekhuyse et al. (1985) Light induced shift and binding of S-antigen in retinal rods. Curr Eye Res 4, 613-618; Philp et al. (1987) Light-stimulated protein movement in rod photoreceptor cells of the rat retina. FEBS Lett 225, 127-132; Whelan et al. (1988) Light-dependent subcellular movement of photoreceptor proteins. J Neurosci Res 20, 263-270; and Zhu et al. (2002) Mouse cone arrestin expression pattern: light induced translocation in cone photoreceptors. Mol Vis 8, 462-471). This translocation of arrestin-1 means that arrestin-1 down regulates the activity of enolase-1 during the dark when it localizes to the inner segments, and that enolase-1 activity is upregulated in the light when arrestin-1 moves to the outer segments. Since the energetic demands of photoreceptors are highest in the dark when the cyclic nucleotide-gated channels are open and the sodium-potassium pump is at its maximum rate to maintain ionic equilibrium (Kaupp et al. (2002) Cyclic nucleotide-gated ion channels. Physiol Rev 82, 769-824), it is not clear what benefit might be provided by potentially increasing glycolytic activity in the light when enolase-1 quenching is reduced as arrestin-1 translocates to the outer segment. Without wishing to be bound by theory, an increase in glycolytic activity during light may function to provide more lactate to the RPE or Müller glia for their metabolic demands, rather than increasing the energy supply to photoreceptors.

### Example 5. Substitution Analysis of Arrestin-I Inhibition of Enolase-1 Activity

Amino acids 361 and 362 or Arrestin-1 were mutated to various amino acids, including, glycine (E361G and D362G), alanine (E361A and D362A), Serine (E361S and D362S), Threonine (E361T and D362T), Leucine (E361L and D362L), Glutamine (E361Q and D362Q), Phenylalanine (E361F and D362F), and Tryptophan (E361W and D362W). The resultant arrestin-1 variants were then tested for their effect on enolase-1 catalytic activity.

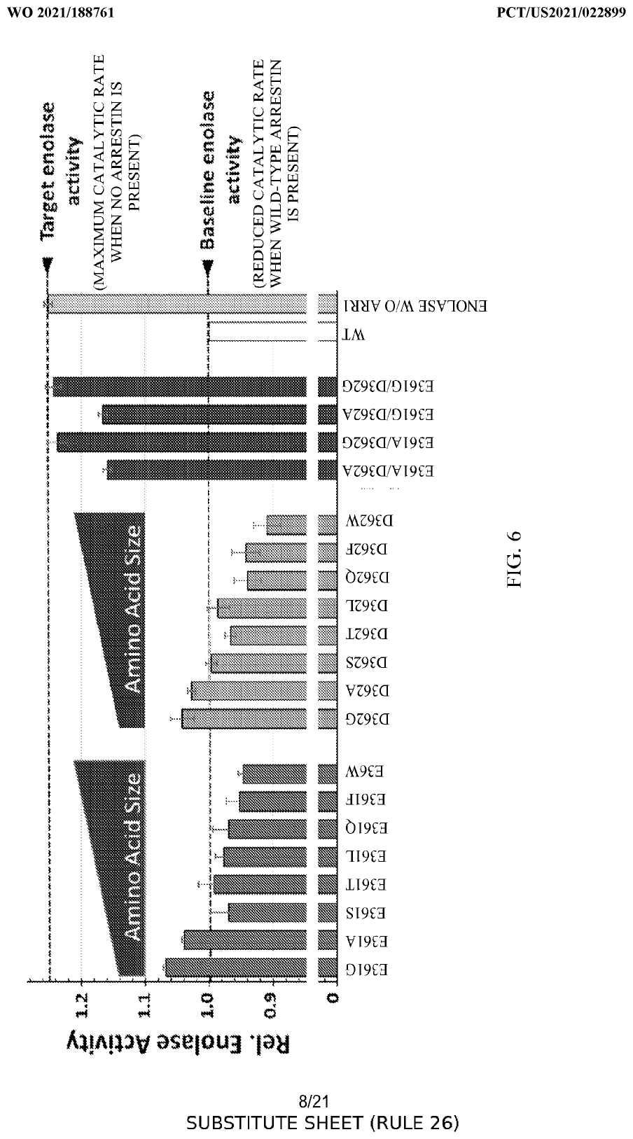

As shown in FIG. 6, as amino acid side chain size increases at each position, there is increasing reduction of enolase catalytic activity. Conversely, reducing the amino acid size to alanine or glycine reduces the inhibiting effect of arrestin-1 on enolase activity. Combining alanine and or glycine substitutions at positions 361 and 362 further reduced the ability of arrestin-1 variants to inhibit enolase activity. E361A/D362G or E361G/D362G arrestin-1 variants have little to no inhibitory effect on enolase activity.

### Example 6 Delivery of Arrestin-1 Variants to Eye

The GEAr1 modified arrestin-1 was packaged into adeno-associated virus (AAV) as described in Zolotukhin et al. 2002 (Production and purification of serotype 1, 2, and 5 recombinant adeno-associated viral vectors. Methods 2002, 28:158-167). AAV containing other arrestin-1 variants can be made in a similar manner.

The GEAr1 modified arrestin-1 was delivered subretinally to rod and cone photoreceptor cells in the mouse as follows: Eyes of mice were dilated with Tropi-Phen (phenylephrine HCL 2.5%, Tropicamide 1% Ophthalmic Solution; Pine Pharmaceuticals, Tonawanda, N.Y., USA) 15 minutes before injection. Transcorneal subretinal injections were performed with a 33-gauge blunt-end needle attached on a 5-mL Hamilton syringe. First, an entering puncture was introduced at the edge of the cornea with a 30-gauge disposable needle, then 1 μL of viral vector mixed with fluorescein dye (0.01% final concentration) was injected through the corneal opening and delivered into the subretinal. An injection was considered successful if it detached at least 80% of the retina visualized by the fluorescence bleb that was monitored by video camera attached to the injection scope allowing real-time assessment of surgical procedures.

### Example 7. GEAr1 Arrestin Gene Therapy Results in Increase NADH and ATP Levels in Rods and Cones

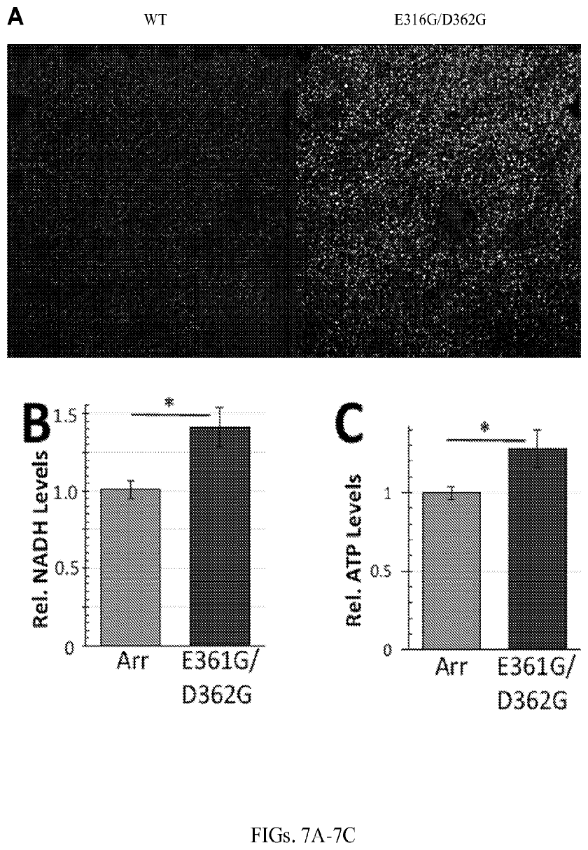

Following subretinal injection, retinal flatmounts of wild-type C57bl/6j injected with AAV containing either wild-type (WT) arrestin or GEAr1 arrestin (E361G/D362G) were visualized with two-photon microscopy to image levels of NADH (reduced nicotinamide adenine dinucleotide), a product of glycolysis. In FIG. 7A, white spots are each individual rod and cone photoreceptor. The brighter white spots in the E361G/D362G eye indicates higher level of NADH production. The mice receiving the GEAr1 arrestin had substantially higher levels of NADH in the rods and cones (see FIG. 7A). As shown in FIG. 7B, retinas from mice injected with GEAr1 arrestin (E361G/D362G) AAV exhibited higher levels of NADH. ATP levels were measured from homogenates of isolate retinas. Retina homogenates from mice injected with GEAr1 arrestin (E361G/D362G) AAV had higher ATP levels than retina homogenates from mice injected with wild-type arrestin (Arr) AAV (FIG. 7C).

### Example 8. GEAr1 Arrestin Gene Therapy Results in Decreased Photoreceptor Loss in Aging Mice

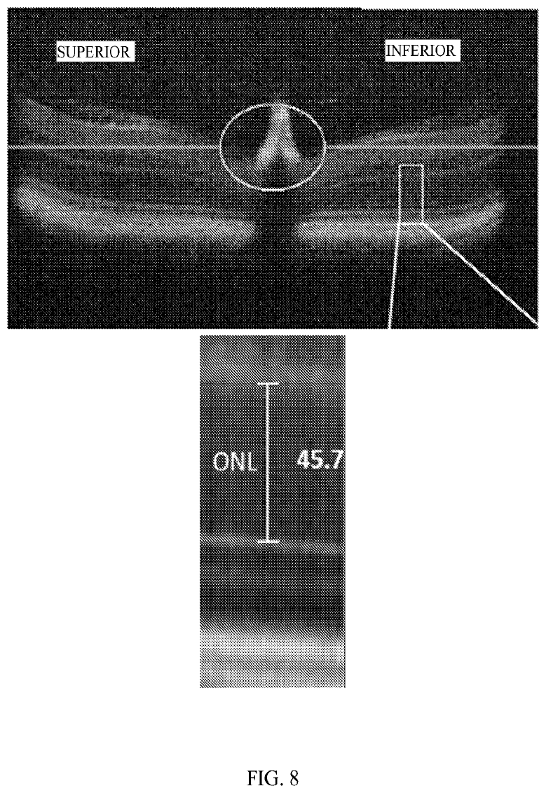

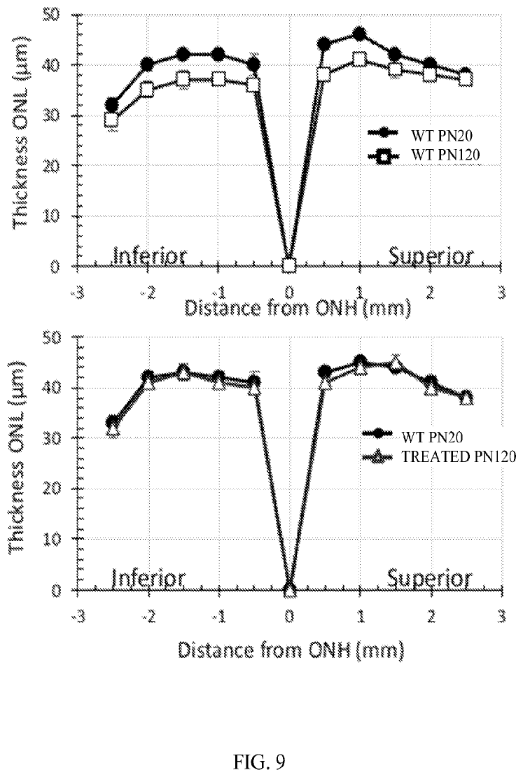

Optical coherence tomography (OCT) of the retinal layers in C57bl/6j mice showed a typical optical section of the retina and outer nuclear layer (ONL) that is measured as an indicator of rod and cone survival (FIG. 8). The graph (FIG. 9) show the ONL thickness from the optic nerve head (ONH) in an untreated wild-type (WT) mouse as it ages from Day 20 (PN20) to Day 120 (PN120) (blue curve). The upper graph (FIG. 9) shows a small loss of photoreceptors that is typical in mice as they age. In mice treated with the modified GEAr1 arrestin, there was no damage associated with the treatment. Further, the mice did not exhibit a significant loss in photoreceptors by 120 (FIG. 9, lower graph).

### Example 9. GEAr1 Arrestin Show Improves Function in Retinitis Pigmentosa

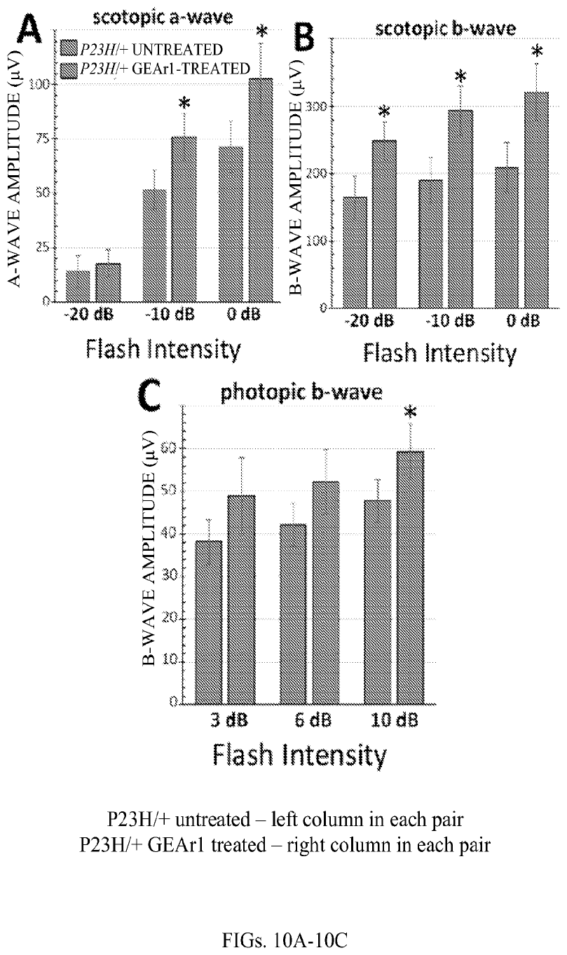

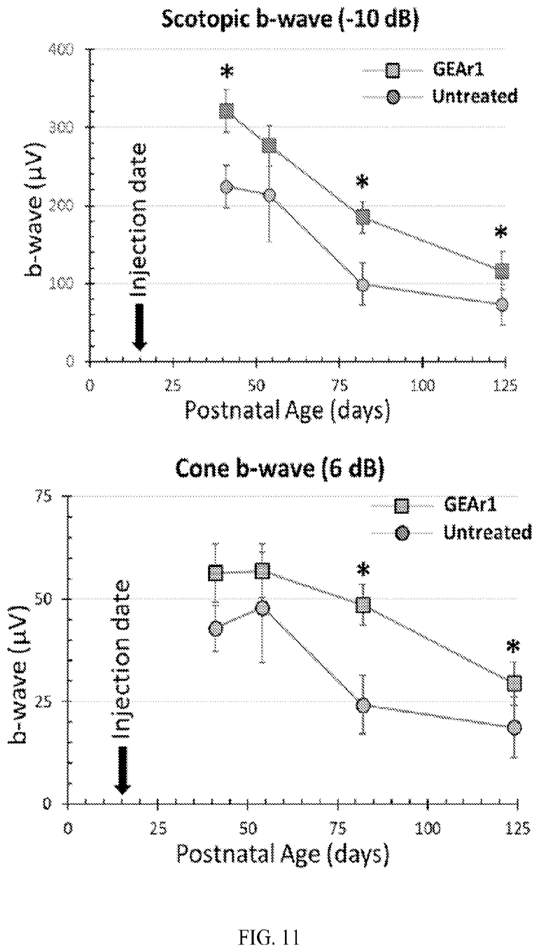

P23H heterozygous mouse model of retinitis pigmentosa were treated with buffer in one eye and either GEAr1 arrestin or wild-type arrestin in the other eye. Electroretinographic (ERG) function was then measured. Mice treated with GEAr1 arrestin showed improved responses of the ERG (orange bars) in both rod photoreceptors (FIG. 10A-10B) and cone photoreceptors (FIG. 10C). Although retina response declined with age in GEAr1 arrestin treated mice, the level of retina response in both rods and cones remained higher at all time points when compared to untreated mice (FIG. 11). The decline was likely due to the P23H mutation in rhodopsin.

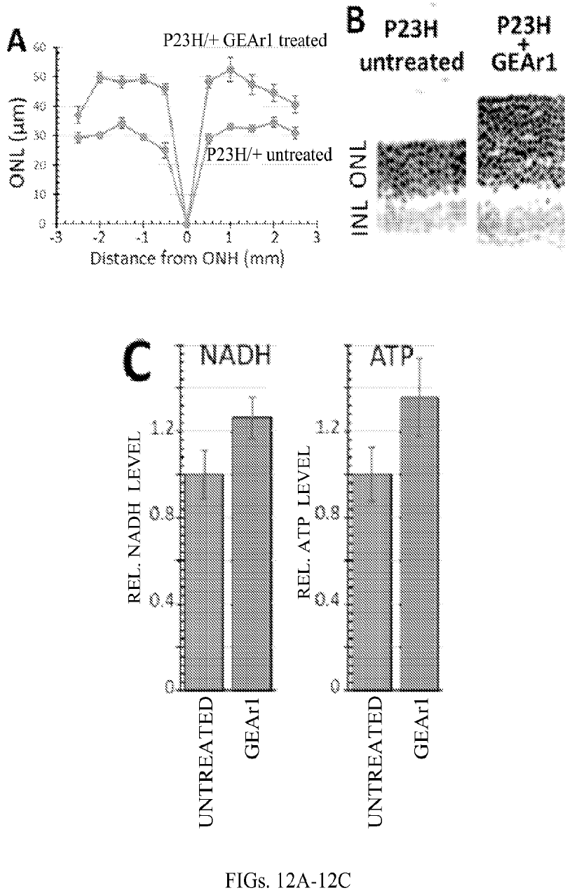

Histological measurement of photoreceptor survival in the untreated and GEAr1-treated P23H mice demonstrated that GEAr1 arrestin treated P23H mice had persistent survival of rod and cone cell bodies (FIG. 12A-12B). NADP and ATP levels were also increased in GEAr1 arrestin treated P23H mice, consistent with the GEAr1 arrestin treated mice having increased enolase activity (FIG. 12C).

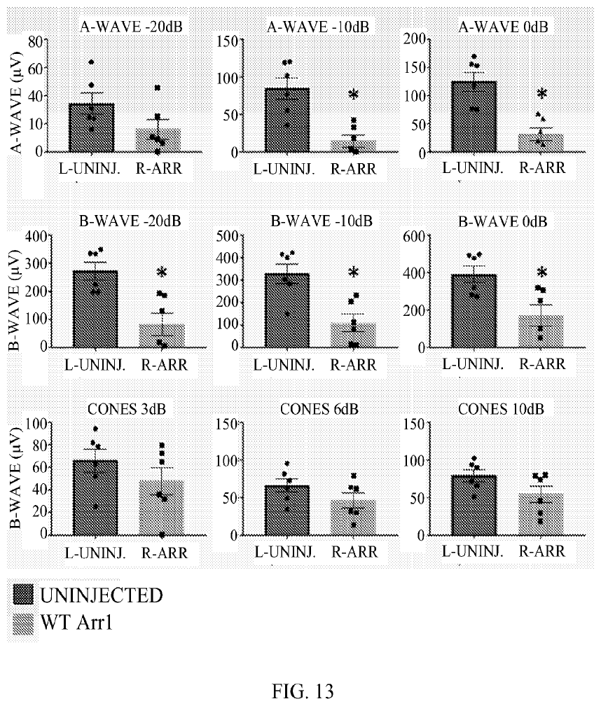

Treatment of the P23 Mouse model of Retinitis Pigmentosa with unmodified arrestin-1 was not protective to the retina. Injection of the P23H mice with unmodified arrestin-1 (WT) AAV did not lead to the same protective effect as the GEAr1 modified arrestin-1 (FIG. 13).

### Example 10. GEAr1 Expression In Vivo Increases Glycolysis

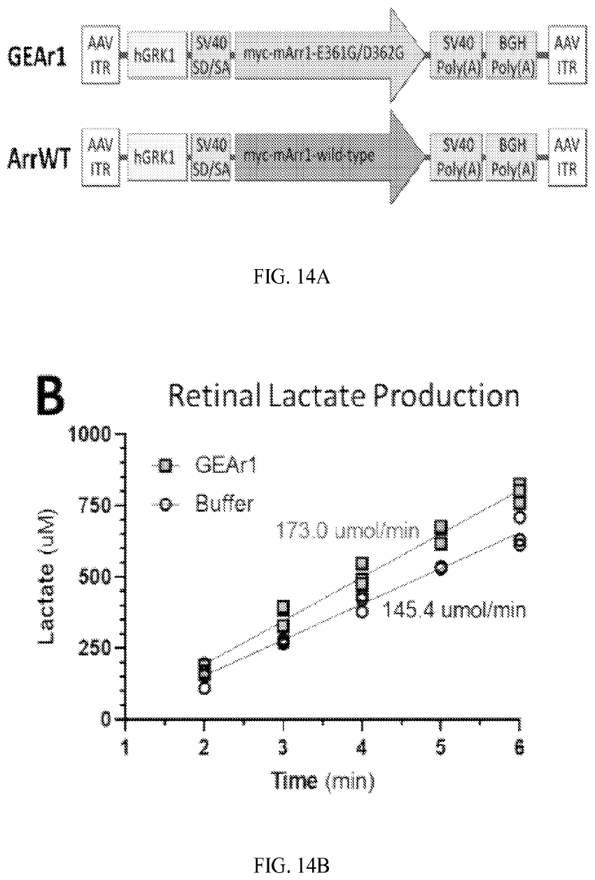

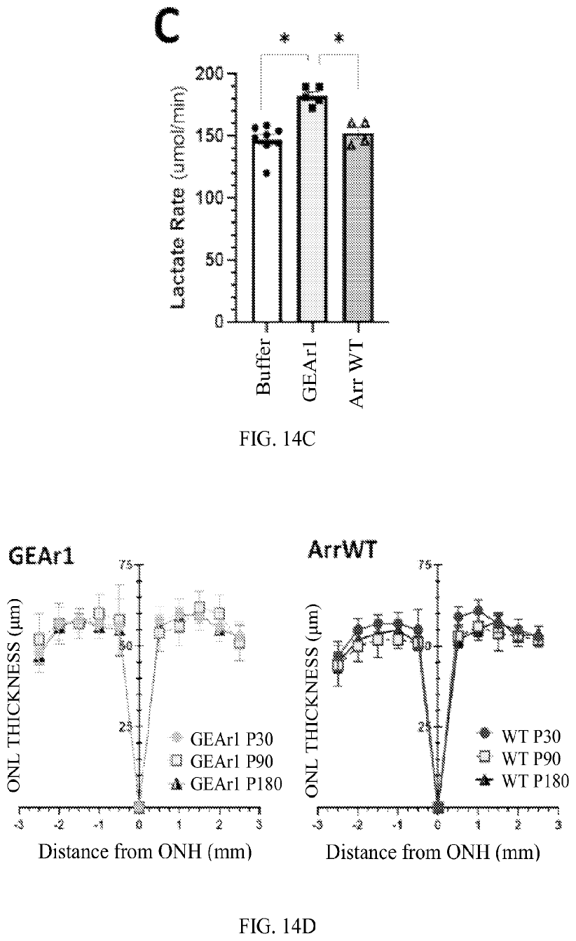

GEAr1 having a myc tag on the C-terminus (for tracing transgene expression) was cloned into the adeno-associated virus (AAV) plasmid pTR-hGRK1 (Beltran W A et al. “rAAV2/5 gene-targeting to rods: dose-dependent efficiency and complications associated with different promoters. Gene Therapy 2010 17:1162), under the control of the human rhodopsin kinase promoter. The rhodopsin kinase promoter selectively drives expression in both rods and cones (Khani S C et al. “AAV-mediated expression targeting of rod and cone photoreceptors with a human rhodopsin kinase promoter. Invest. Ophthalmol Vis Sci 2007 48:3954-3961 and Beltran W A et al. “Optimization of retinal gene therapy for X-Linked retinitis pigmentosa due to RPGR mutations. Molecular Therapy 2017 25:1866-1880) (FIG. 14A). This plasmid was used to package GEAr1 into AAV2(quad Y-F+T-V) capsid serotype (Mowat F M et al. “Tyrosine capsid-mutant AAV vectors for gene delivery to the canine retina from a subretinal or intravitreal approach.” Gene therapy 2014 21:96-105) at 6.5×10¹² vg/mL (viral genomes per mL). A similar AAV vector waw made packaging unmodified arrestin1 construct (ArrWT) as a control (4.7×10¹² vg/mL). The modified arrestin1 (GEAr1) was delivered by subretinal injection to C57BL/6 mice Control mice received either the wild-type Arr1 AAV or saline.

To measure if there was any effect on glycolytic rate, the retina was removed from treated mice 30 days post-injection, placed in Kreb's Ringer buffer, and replicate samples of the buffer collected at one-minute intervals. Lactate in the samples were then measured as an estimate of glycolytic output (Lactate-Glo, Promega; Miranda C J et al. An arrestin-1 surface opposite of its interface with photoactivated rhodopsin engages with enolase-1.” J Biol Chem 2020 295:6498-6508) and rate of lactate production derived by regression analysis (FIG. 14B). Retinas from mice injected with AAV-GEAr1 demonstrated a 24% higher lactate production rate than mice receiving AAV-ArrWT or buffer (FIG. 14C). GEAr1 was found to increase glycolysis in vivo, particularly in the background of a full complement of endogenous arrestin-1.

The capacity of the retina to maintain increased glucose consumption was analyzed. If the eye has a relatively finite supply of glucose, then more rapid consumption of glucose could be detrimental, leading to a loss of photoreceptors as available glucose becomes depleted. However, if the increased glucose demand can be met, then increased glycolysis should not be damaging. AAV-GEAr1 or ArrWT was delivered subretinally to another cohort of C57BL/6 mice at P30 (post-natal day 30). Outer nuclear layer thickness (ONL) was then measured by OCT out to P180 (FIG. 14D). In mice treated with the GEAr1, there is no evidence of decline in ONL thickness up to six months, indicating that up-regulation of glycolysis mediated by the modified arrestin-1 is not damaging to the retina, and overexpression of GEAr1 is not detrimental to photoreceptor survival.

### Example 11. GEAr2 Decreases Retinal Degeneration

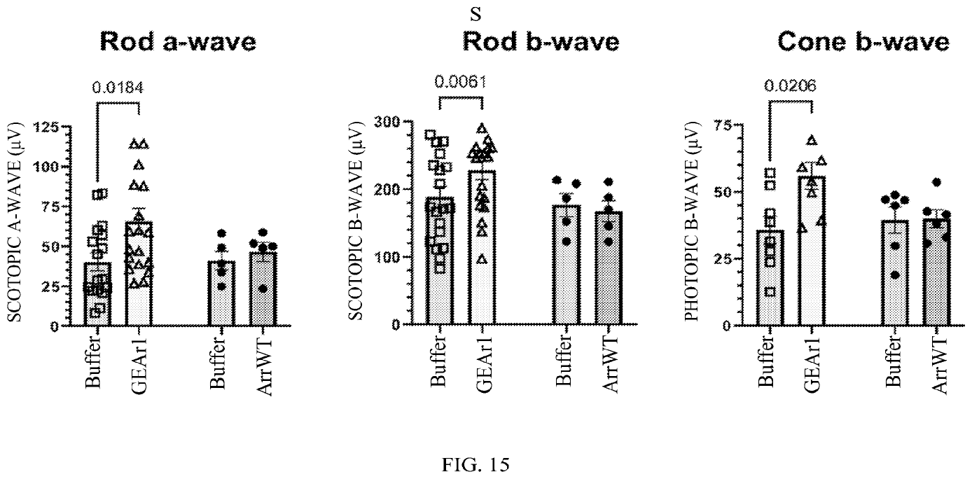

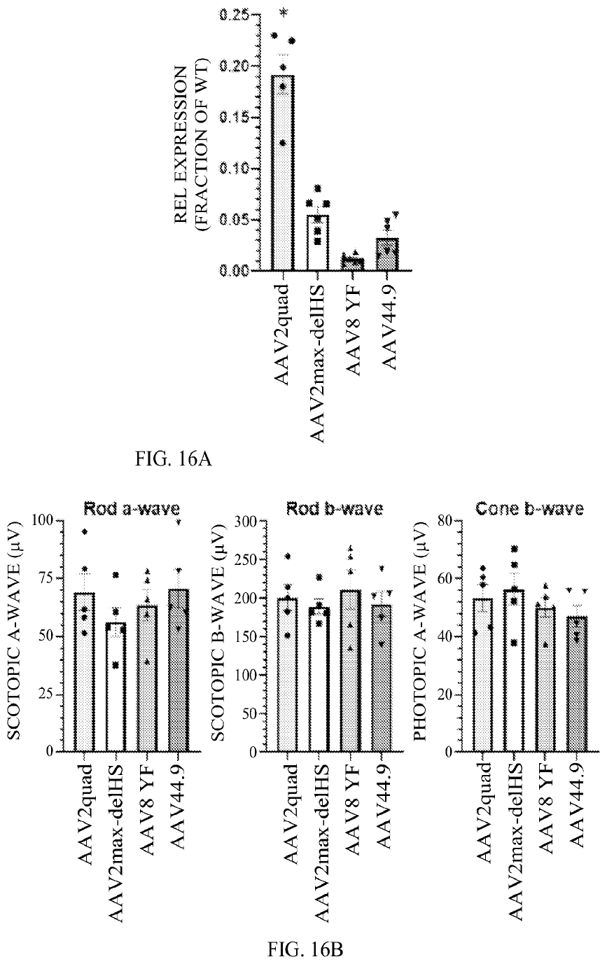

A mouse model of retinal degeneration was used to determine if increased glycolytic function could be protective for the photoreceptors. P23H heterozygous knock-in mouse model (P23H+/−) (RhoP23H/+, Jackson Stock No: 017628; Sakami S et al. “Probing mechanisms of photoreceptor degeneration in a new mouse model of the common form of autosomal dominant retinitis pigmentosa due to P23H opsin mutations.” J Biol Chem 2011 86:10551-10567) was used because of its moderate rate of retinal degeneration and high level of characterization (Sakami S et al. “Probing mechanisms of photoreceptor degeneration in a new mouse model of the common form of autosomal dominant retinitis pigmentosa due to P23H opsin mutations.” J Biol Chem 2011 86:10551-10567; Chiang W-C et al. “Robust endoplasmic reticulum-associated degradation of rhodopsin precedes retinal degeneration.” Molecular Neurobiology 2015 52:679-695; Comitato A et al. “Dominant and recessive mutations in rhodopsin activate different cell death pathways.” Human Molecular Genetics 2016 25:2801-2812; Liu H et al. “Photoreceptor cells influence retinal vascular degeneration in mouse models of retinal degeneration and diabetes.” Invest Ophthalmol Vis Sci 2016 57:4272-4281; Mitra R N et al. “Genomic form of rhodopsin DNA nanoparticles rescued autosomal dominant Retinitis pigmentosa in the P23H knock-in mouse model.” Biomaterials 2018 157:26-39; and Nakamura P A et al. “Small molecule Photoregulin3 prevents retinal degeneration in the RhoP23H mouse model of retinitis pigmentosa.” eLife 2017 6:e30577). Mice were injected subretinally at P15 with the AAV-GEAr1 or AAV-ArrWT in one eye and buffer in the contralateral eye. Mice expressing GEAr1 showed significant preservation of both scotopic and photopic electroretinography (ERG) function when measured 30 days post injection (P45). Mice expressing heterologous ArrWT did not exhibit preservation of scotopic and photopic ERG function (FIG. 15).

### Example 12. Expression of GEAr1 in Retina Following Delivery Using Different AAV Vectors

A GEAr1 expression cassette, hGRK1-myc-GEAr1-2A-GFP, was packaged into four different AAV vectors having different capsid serotypes. The AAV vectors used were: AAV2(quad Y-F+T-V), AAV2max-delHS, AAV8 Y733F, and AAV44.9(E5331D) (as described in Mowat et al. 2014; Boye S L et al. “Impact of heparan sulfate binding on transduction of retina by recombinant adeno-associated virus vectors.” J Virol. 2016 90:4215-4231; De Silva S R et al. “Single residue AAV capsid mutation improves transduction of photoreceptors in the Abca4(−/−) mouse and bipolar cells in the rd1 mouse and human retina ex-vivo.” Gene therapy 2016 23:767-774; and Boye S L et al. “Novel AAV44.9-based vectors display exceptional characteristics for retinal gene therapy.” Molecular Therapy 2020 Jun. 3; 28:1464-1478 (Epub Apr. 11, 2020), respectively). The AAV Vectors were administered to C57BL/6J mice at P30 via an intravitreal route of delivery since subretinal injections led to a wide range of variability in ERG function (likely due to injection damage in the P23H degenerating retina). Expression level was determined by Western blot analysis of retinas probed with anti-myc. AAV2(quad Y-F+T-V) drove the best expression of the transgene (FIG. 16A). ERG function at P60 was not negatively impacted by expression of GEAr1 in either rods or cones (FIG. 16B). AAV2(quad Y-F+T-V) (AAV2quad) was used for further experiments.

### Example 13. Expression of GEAr1 in Retina Using Different Promoters

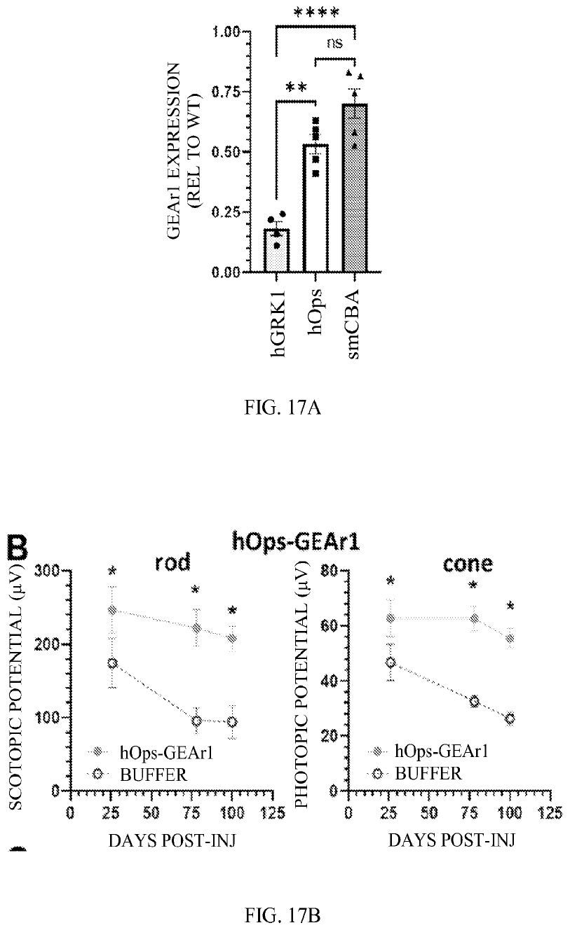

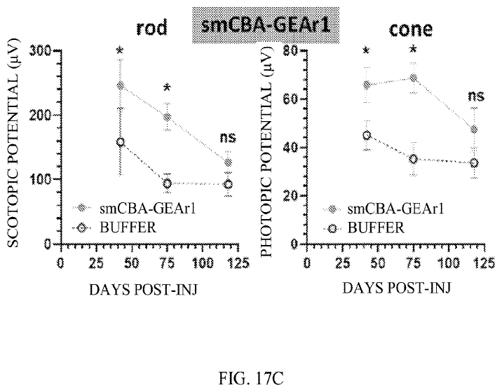

A GEAr1 expression cassettes were generated in which the GEAr2 coding sequence was operably linked to different promoters. The promoters used were the rhodopsin kinase promoter (hGRK1) for driving moderate levels of photoreceptor-specific expression (Beltran et al. 2010), a modified chicken β-actin promoter (smCBA) for driving high levels of ubiquitous expression (Boye S L et al. (2006) Transduction and tropism of an abbreviated form of CMV-chicken β-actin promoter (CBA) with AAV in mouse retina. Invest Ophthalmol Vis Sci 2006 47:852-852), and a truncated human opsin promoter (hOps) for driving high levels of rod photoreceptor-specific expression (Lee J et al. (2010) Quantitative fine-tuning of photoreceptor cis-regulatory elements through affinity modulation of transcription factor binding sites. Gene Ther 2010 17:1390-9). GEAr1 was cloned behind each of these promoters and packaged into AAV2quad (10⁹ vg/eye) and delivered intravitreally into a cohort of C57BL/6J mice at P30. Eyes were collected at P60 for analysis. In these mice, both hOps and smCBA drove the highest level of expression, reaching more than 50% of the level of the endogenous arrestin1 (FIG. 17A).

AAV-hOps-GEAr2 and AAV-smCBA-GEAr2 were then delivered intravitreally to P23H+/− mice. ERG function was measured at three time points up to 100 days after injection to analyze the potential of GEAr1 in preserving ERG function in this inherited retinal disease (IRD) model. IRD mice receiving either smCBA-GEAr1 or hOps-GEAr1 exhibited significant initial preservation of ERG function compared to the buffer-treated controls (FIG. 17B and FIG. 17C). Preservation of ERG function declined by four months in smCBA-GEAr1 treated mice (FIG. 17C). In contrast, IRD mice treated with the hOps-driven GEAr1 preservation of ERG function in both rods and cones persisted to 3.5 months (FIG. 17B).

TABLE 5
Sequences

SEQ ID NO: 1. mouse wild-type arrestin-1 with His and myc tags; nucleic acid sequence
atgcaccaccaccaccaccacaccatggcagcctgcgggaagaccaataaatcccacgtcatcttcaagaagg
tctctcgggacaagtcggtgaccatctacctggggaagagagactatgtagaccacgtcagccaagtagagcc
tgtagatggtgttgtgctggtggaccctgagcttgtgaaggggaagaaggtgtatgtcaccctgacctgcgcc
ttccgctatggccaggaggacattgatgtgatgggcctgaccttccgcagggacctgtatttttctcgggtcc
aggtataccctcctgttggggccatgagtgtcctcacccaactccaagagagcctgctcaagaaactgggaga
caacacgtacccctttctgctcacgtttcctgactacctaccctgttcagtgatgttgcagccagctccccag
gatgtaggcaagagctgtggggttgactttgaggttaaagcatttgcctcagacatcacagatcctgaggagg
acaagatccccaagaagagctctgtgcggttactgatccggaaagtgcagcatgccccacctgagatgggtcc
ccagcccagtgctgaagcctcctggcagttcttcatgtctgacaagcccctgaacctctcagtctccctcagc
aaagagatatatttccatggggagcccatccccgtgactgtgaccgtgaccaacaacaccgacaaggttgtga
agaagattaaagtatcagtggaacagatagccaatgtggttctctactcaagtgactattacgtcaagcctgt
agcctcggaggaaacacaagaaaaagtgcagccaaacagcactttgaccaagacactggtgctggtgccccta
ctggctaacaacagagaaagaagaggcatcgcgctggatgggaagatcaagcatgaggacacaaacctggcct
ccagcaccatcatcaaggagggcatcgaccggaccgtcatggggatcctggtgtcataccatatcaaggtgaa
gctcacggtgtcgggctttctaggagagctcacatccagtgaagtggctaccgaggtgccgttccgcctcatg
cacccgcagcctgaggatccagcaaaagaaagtgttcaggatgaaaatttggtttttgaagagtttgctcgcc
aaaatctgaaagatactggagagaacacagaagggaagaaagatgaggatgctggccaggatgaggagcaaaa
acttatctctgaggaagatctg
SEQ ID NO: 2. mouse wild-type arrestin-1 with His and myc tags; amino acid sequence
MHHHHHHTMAACGKTNKSHVIFKKVSRDKSVTIYLGKRDYVDHVSQVEPVDGVVLVDPELVKGKKVYVTLTCA
FRYGQEDIDVMGLTFRRDLYFSRVQVYPPVGAMSVLTQLQESLLKKLGDNTYPFLLTFPDYLPCSVMLQPAPQ
DVGKSCGVDFEVKAFASDITDPEEDKIPKKSSVRLLIRKVQHAPPEMGPQPSAEASWQFFMSDKPLNLSVSLS
KEIYFHGEPIPVTVTVTNNTDKVVKKIKVSVEQIANVVLYSSDYYVKPVASEETQEKVQPNSTLTKTLVLVPL
LANNRERRGIALDGKIKHEDTNLASSTIIKEGIDRTVMGILVSYHIKVKLTVSGFLGELTSSEVATEVPFRLM
HPQPEDPAKESVQDENLVFEEFARQNLKDTGENTEGKKDEDAGQDEEQKLISEEDL
SEQ ID NO: 3. mouse wild-type arrestin-1; nucleic acid sequence
atggcagcctgcgggaagaccaataaatcccacgtcatcttcaagaaggtctctcgggacaagtcggtgacca
tctacctggggaagagagactatgtagaccacgtcagccaagtagagcctgtagatggtgttgtgctggtgga
ccctgagcttgtgaaggggaagaaggtgtatgtcaccctgacctgcgccttccgctatggccaggaggacatt
gatgtgatgggcctgaccttccgcagggacctgtatttttctcgggtccaggtataccctcctgttggggcca
tgagtgtcctcacccaactccaagagagcctgctcaagaaactgggagacaacacgtacccctttctgctcac
gtttcctgactacctaccctgttcagtgatgttgcagccagctccccaggatgtaggcaagagctgtggggtt
gactttgaggttaaagcatttgcctcagacatcacagatcctgaggaggacaagatccccaagaagagctctg
tgcggttactgatccggaaagtgcagcatgccccacctgagatgggtccccagcccagtgctgaagcctcctg
gcagttcttcatgtctgacaagcccctgaacctctcagtctccctcagcaaagagatatatttccatggggag
cccatccccgtgactgtgaccgtgaccaacaacaccgacaaggttgtgaagaagattaaagtatcagtggaac
agatagccaatgtggttctctactcaagtgactattacgtcaagcctgtagcctcggaggaaacacaagaaaa
agtgcagccaaacagcactttgaccaagacactggtgctggtgcccctactggctaacaacagagaaagaaga
ggcatcgcgctggatgggaagatcaagcatgaggacacaaacctggcctccagcaccatcatcaaggagggca
tcgaccggaccgtcatggggatcctggtgtcataccatatcaaggtgaagctcacggtgtcgggctttctagg
agagctcacatccagtgaagtggctaccgaggtgccgttccgcctcatgcacccgcagcctgaggatccagca
aaagaaagtgttcaggatgaaaatttggtttttgaagagtttgctcgccaaaatctgaaagatactggagaga
acacagaagggaagaaagatgaggatgctggccaggatgag
SEQ ID NO: 4. mouse wild-type arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPEDPA
KESVQDENLVFEEFARQNLKDTGENTEGKKDEDAGQDEDAGQDE
SEQ ID NO: 5. GEAr1 variant of mouse arrestin-1 with His and myc tags; nucleic acid
sequence
atgcaccaccaccaccaccacaccatggcagcctgcgggaagaccaataaatcccacgtcatcttcaagaagg
tctctcgggacaagtcggtgaccatctacctggggaagagagactatgtagaccacgtcagccaagtagagcc
tgtagatggtgttgtgctggtggaccctgagcttgtgaaggggaagaaggtgtatgtcaccctgacctgcgcc
ttccgctatggccaggaggacattgatgtgatgggcctgaccttccgcagggacctgtatttttctcgggtcc
aggtataccctcctgttggggccatgagtgtcctcacccaactccaagagagcctgctcaagaaactgggaga
caacacgtacccctttctgctcacgtttcctgactacctaccctgttcagtgatgttgcagccagctccccag
gatgtaggcaagagctgtggggttgactttgaggttaaagcatttgcctcagacatcacagatcctgaggagg
acaagatccccaagaagagctctgtgcggttactgatccggaaagtgcagcatgccccacctgagatgggtcc
ccagcccagtgctgaagcctcctggcagttcttcatgtctgacaagcccctgaacctctcagtctccctcagc
aaagagatatatttccatggggagcccatccccgtgactgtgaccgtgaccaacaacaccgacaaggttgtga
agaagattaaagtatcagtggaacagatagccaatgtggttctctactcaagtgactattacgtcaagcctgt
agcctcggaggaaacacaagaaaaagtgcagccaaacagcactttgaccaagacactggtgctggtgccccta
ctggctaacaacagagaaagaagaggcatcgcgctggatgggaagatcaagcatgaggacacaaacctggcct
ccagcaccatcatcaaggagggcatcgaccggaccgtcatggggatcctggtgtcataccatatcaaggtgaa
gctcacggtgtcgggctttctaggagagctcacatccagtgaagtggctaccgaggtgccgttccgcctcatg
cacccgcagcctggaggtccagcaaaagaaagtgttcaggatgaaaatttggtttttgaagagtttgctcgcc
aaaatctgaaagatactggagagaacacagaagggaagaaagatgaggatgctggccaggatgaggagcaaaa
acttatctctgaggaagatctg
SEQ ID NO: 6. GEAr1 variant of mouse arrestin-1 with His and myc tags; amino acid
sequence
MHHHHHHTMAACGKTNKSHVIFKKVSRDKSVTIYLGKRDYVDHVSQVEPVDGWLVDPELVKGKKVYVTLTCA
FRYGQEDIDVMGLTFRRDLYFSRVQVYPPVGAMSVLTQLQESLLKKLGDNTYPFLLTFPDYLPCSVMLQPAPQ
DVGKSCGVDFEVKAFASDITDPEEDKIPKKSSVRLLIRKVQHAPPEMGPQPSAEASWQFFMSDKPLNLSVSLS
KEIYFHGEPIPVTVTVTNNTDKWKKIKVSVEQIANWLYSSDYYVKPVASEETQEKVQPNSTLTKTLVLVPL
LANNRERRGIALDGKIKHEDTNLASSTIIKEGIDRTVMGILVSYHIKVKLTVSGFLGELTSSEVATEVPFRLM
HPQPGGPAKESVQDENLVFEEFARQNLKDTGENTEGKKDEDAGQDEEQKLISEEDL
SEQ ID NO: 7. GEAr1 variant of mouse arrestin-1; nucleic acid sequence
atggcagcctgcgggaagaccaataaatcccacgtcatcttcaagaaggtctctcgggacaagtcggtgacca
tctacctggggaagagagactatgtagaccacgtcagccaagtagagcctgtagatggtgttgtgctggtgga
ccctgagcttgtgaaggggaagaaggtgtatgtcaccctgacctgcgccttccgctatggccaggaggacatt
gatgtgatgggcctgaccttccgcagggacctgtatttttctcgggtccaggtataccctcctgttggggcca
tgagtgtcctcacccaactccaagagagcctgctcaagaaactgggagacaacacgtacccctttctgctcac
gtttcctgactacctaccctgttcagtgatgttgcagccagctccccaggatgtaggcaagagctgtggggtt
gactttgaggttaaagcatttgcctcagacatcacagatcctgaggaggacaagatccccaagaagagctctg
tgcggttactgatccggaaagtgcagcatgccccacctgagatgggtccccagcccagtgctgaagcctcctg
gcagttcttcatgtctgacaagcccctgaacctctcagtctccctcagcaaagagatatatttccatggggag
cccatccccgtgactgtgaccgtgaccaacaacaccgacaaggttgtgaagaagattaaagtatcagtggaac
agatagccaatgtggttctctactcaagtgactattacgtcaagcctgtagcctcggaggaaacacaagaaaa
agtgcagccaaacagcactttgaccaagacactggtgctggtgcccctactggctaacaacagagaaagaaga
ggcatcgcgctggatgggaagatcaagcatgaggacacaaacctggcctccagcaccatcatcaaggagggca
tcgaccggaccgtcatggggatcctggtgtcataccatatcaaggtgaagctcacggtgtcgggctttctagg
agagctcacatccagtgaagtggctaccgaggtgccgttccgcctcatgcacccgcagcctggaggtccagca
aaagaaagtgttcaggatgaaaatttggtttttgaagagtttgctcgccaaaatctgaaagatactggagaga
acacagaagggaagaaagatgaggatgctggccaggatgag
SEQ ID NO: 8. GEAr1 variant of mouse arrestin-1; nucleic acid sequence
atggcagcctgcgggaagaccaataaatcccacgtcatcttcaagaaggtctctcgggacaagtcggtgacca
tctacctggggaagagagactatgtagaccacgtcagccaagtagagcctgtagatggtgttgtgctggtgga
ccctgagcttgtgaaggggaagaaggtgtatgtcaccctgacctgcgccttccgctatggccaggaggacatt
gatgtgatgggcctgaccttccgcagggacctgtatttttctcgggtccaggtataccctcctgttggggcca
tgagtgtcctcacccaactccaagagagcctgctcaagaaactgggagacaacacgtacccctttctgctcac
gtttcctgactacctaccctgttcagtgatgttgcagccagctccccaggatgtaggcaagagctgtggggtt
gactttgaggttaaagcatttgcctcagacatcacagatcctgaggaggacaagatccccaagaagagctctg
tgcggttactgatccggaaagtgcagcatgccccacctgagatgggtccccagcccagtgctgaagcctcctg
gcagttcttcatgtctgacaagcccctgaacctctcagtctccctcagcaaagagatatatttccatggggag
cccatccccgtgactgtgaccgtgaccaacaacaccgacaaggttgtgaagaagattaaagtatcagtggaac
agatagccaatgtggttctctactcaagtgactattacgtcaagcctgtagcctcggaggaaacacaagaaaa
agtgcagccaaacagcactttgaccaagacactggtgctggtgcccctactggctaacaacagagaaagaaga
ggcatcgcgctggatgggaagatcaagcatgaggacacaaacctggcctccagcaccatcatcaaggagggca
tcgaccggaccgtcatggggatcctggtgtcataccatatcaaggtgaagctcacggtgtcgggctttctagg
agagctcacatccagtgaagtggctaccgaggtgccgttccgcctcatgcacccgcagcctggnggnccagca
aaagaaagtgttcaggatgaaaatttggtttttgaagagtttgctcgccaaaatctgaaagatactggagaga
acacagaagggaagaaagatgaggatgctggccaggatgag
SEQ ID NO: 9. GEAr1 variant of mouse arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPGGPA
KESVQDENLVFEEFARQNLKDTGENTEGKKDEDAGQDE
SEQ ID NO: 10 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPAAPA
KESVQDENLVFEEFARQNLKDTGENTEGKKDEDAGQDE
SEQ ID NO: 11 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPAGPA
KESVQDENLVFEEFARQNLKDTGENTEGKKDEDAGQDE
SEQ ID NO: 12 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPGAPA
KESVQDENLVFEEFARQNLKDTGENTEGKKDEDAGQDE
SEQ ID NO: 13 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPGDPA
KESVQDENLVFEEFARQNLKDTGENTEGKKDEDAGQDE
SEQ ID NO: 14 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPADPA
KESVQDENLVFEEFARQNLKDTGENTEGKKDEDAGQDE
SEQ ID NO: 15 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPEAPA
KESVQDENLVFEEFARQNLKDTGENTEGKKDEDAGQDE
SEQ ID NO: 16 mouse variant arrestin-1; amino acid sequence
MAACGKTNKSHVIFKKVSRDKSVTIYLGKRDYVDHVSQVEPVDGVVLVDPELVKGKKVYVTLTCAFRYGQEDI
DVMGLTFRRDLYFSRVQVYPPVGAMSVLTQLQESLLKKLGDNTYPFLLTFPDYLPCSVMLQPAPQDVGKSCGV
DFEVKAFASDITDPEEDKIPKKSSVRLLIRKVQHAPPEMGPQPSAEASWQFFMSDKPLNLSVSLSKEIYFHGE
PIPVTVTVTNNTDKVVKKIKVSVEQIANVVLYSSDYYVKPVASEETQEKVQPNSTLTKTLVLVPLLANNRERR
GIALDGKIKHEDTNLASSTIIKEGIDRTVMGILVSYHIKVKLTVSGFLGELTSSEVATEVPFRLMHPQPEGPA
KESVQDENLVFEEFARQNLKDTGENTEGKKDEDAGQDE
SEQ ID NO: 17. Human wild-type arrestin-1; nucleic acid sequence
atggcagccagcgggaagaccagcaagtccgaaccgaaccatgttatcttcaagaagatctcccgggacaaat
cggtgaccatctacctggggaacagagactacatagaccatgtcagccaagtccagcctgtggatggtgtcgt
gttggttgatcctgatcttgtgaagggaaagaaagtgtatgtcactctgacctgcgccttccgctatggccaa
gaggacattgacgtgatcggcttgaccttccgcagggacctgtacttctcccgggtccaggtgtatcctcctg
tgggggccgcgagcacccccacaaaactgcaagagagcctgcttaaaaagctggggagcaacacgtacccctt
tctcctgacgtttcctgactacttgccctgttcagtgatgttgcagccagctccacaagattcagggaagtcc
tgtggggttgactttgaggtcaaagcattcgccacagacagcaccgatgccgaagaggacaaaatccccaaga
agagctccgtgcgattactgatccgcaaagtacagcatgccccacttgagatgggtccccagccccgagctga
ggcggcctggcagttcttcatgtctgacaagcccctgcaccttgcggtctctctcaacaaagagatctatttc
catggggagcccatccctgtgaccgtgactgtcaccaataacacagagaagaccgtgaagaagattaaagcat
tcgtggaacaggtggccaatgtggttctctactcgagtgattattacgtcaagcccgtggctatggaggaagc
gcaagaaaaagtgccaccaaacagcactttgaccaagacgctgacgctgctgcccttgctggctaacaatcga
gaaaggagaggcattgccctggatgggaaaatcaagcacgaggacacaaaccttgcctccagcaccatcatta
aggagggcatagaccggaccgtcctgggaatcctggtgtcttaccagatcaaggtgaagctcacagtgtcagg
ctttctgggagagctcacctccagtgaagtcgccactgaggtcccattccgcctcatgcaccctcagcctgag
gacccagctaaggaaagttatcaggatgcaaatttagtttttgaggagtttgctcgccataatctgaaagatg
caggagaagctgaggaggggaagagagacaagaatgacgttgatgag
SEQ ID NO: 18. Human wild-type ortholog of arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTKLQESLLKKLGSNTYPFLLTFPDYLPCSVMLQPAPQDSGKS
CGVDFEVRAFATDSTDAEEDRIPRRSSVRLLIRRVQHAPLEMGPQPRAEAAWQFFMSDRPLHLAVSLNREIYF
HGEPIPVTVTVTNNTEKTVKKIKAFVEQVANVVLYSSDYYVKPVAMEEAQEKVPPNSTLTKTLTLLPLLANNR
ERRGIALDGKIKHEDTNLASSTIIKEGIDRTVLGILVSYQIKVKLTVSGFLGELTSSEVATEVPFRLMHPQPE
DPARESYQDANLVFEEFARHNLKDAGEAEEGRRDRNDVDE
SEQ ID NO: 19. Human GEAr1 variant of arrestin-1; nucleic acid sequence
atggcagccagcgggaagaccagcaagtccgaaccgaaccatgttatcttcaagaagatctcccgggacaaat
cggtgaccatctacctggggaacagagactacatagaccatgtcagccaagtccagcctgtggatggtgtcgt
gttggttgatcctgatcttgtgaagggaaagaaagtgtatgtcactctgacctgcgccttccgctatggccaa
gaggacattgacgtgatcggcttgaccttccgcagggacctgtacttctcccgggtccaggtgtatcctcctg
tgggggccgcgagcacccccacaaaactgcaagagagcctgcttaaaaagctggggagcaacacgtacccctt
tctcctgacgtttcctgactacttgccctgttcagtgatgttgcagccagctccacaagattcagggaagtcc
tgtggggttgactttgaggtcaaagcattcgccacagacagcaccgatgccgaagaggacaaaatccccaaga
agagctccgtgcgattactgatccgcaaagtacagcatgccccacttgagatgggtccccagccccgagctga
ggcggcctggcagttcttcatgtctgacaagcccctgcaccttgcggtctctctcaacaaagagatctatttc
catggggagcccatccctgtgaccgtgactgtcaccaataacacagagaagaccgtgaagaagattaaagcat
tcgtggaacaggtggccaatgtggttctctactcgagtgattattacgtcaagcccgtggctatggaggaagc
gcaagaaaaagtgccaccaaacagcactttgaccaagacgctgacgctgctgcccttgctggctaacaatcga
gaaaggagaggcattgccctggatgggaaaatcaagcacgaggacacaaaccttgcctccagcaccatcatta
aggagggcatagaccggaccgtcctgggaatcctggtgtcttaccagatcaaggtgaagctcacagtgtcagg
ctttctgggagagctcacctccagtgaagtcgccactgaggtcccattccgcctcatgcaccctcagcctgga
ggtccagctaaggaaagttatcaggatgcaaatttagtttttgaggagtttgctcgccataatctgaaagatg
caggagaagctgaggaggggaagagagacaagaatgacgttgatgag
SEQ ID NO: 20. Human GEAr1 variant of arrestin-1; nucleic acid sequence
atggcagccagcgggaagaccagcaagtccgaaccgaaccatgttatcttcaagaagatctcccgggacaaat
cggtgaccatctacctggggaacagagactacatagaccatgtcagccaagtccagcctgtggatggtgtcgt
gttggttgatcctgatcttgtgaagggaaagaaagtgtatgtcactctgacctgcgccttccgctatggccaa
gaggacattgacgtgatcggcttgaccttccgcagggacctgtacttctcccgggtccaggtgtatcctcctg
tgggggccgcgagcacccccacaaaactgcaagagagcctgcttaaaaagctggggagcaacacgtacccctt
tctcctgacgtttcctgactacttgccctgttcagtgatgttgcagccagctccacaagattcagggaagtcc
tgtggggttgactttgaggtcaaagcattcgccacagacagcaccgatgccgaagaggacaaaatccccaaga
agagctccgtgcgattactgatccgcaaagtacagcatgccccacttgagatgggtccccagccccgagctga
ggcggcctggcagttcttcatgtctgacaagcccctgcaccttgcggtctctctcaacaaagagatctatttc
catggggagcccatccctgtgaccgtgactgtcaccaataacacagagaagaccgtgaagaagattaaagcat
tcgtggaacaggtggccaatgtggttctctactcgagtgattattacgtcaagcccgtggctatggaggaagc
gcaagaaaaagtgccaccaaacagcactttgaccaagacgctgacgctgctgcccttgctggctaacaatcga
gaaaggagaggcattgccctggatgggaaaatcaagcacgaggacacaaaccttgcctccagcaccatcatta
aggagggcatagaccggaccgtcctgggaatcctggtgtcttaccagatcaaggtgaagctcacagtgtcagg
ctttctgggagagctcacctccagtgaagtcgccactgaggtcccattccgcctcatgcaccctcagcctggn
ggnccagctaaggaaagttatcaggatgcaaatttagtttttgaggagtttgctcgccataatctgaaagatg
caggagaagctgaggaggggaagagagacaagaatgacgttgatgag
SEQ ID NO: 21. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTRLQESLLRRLGSNTYPFLLTFPDYLPCSVMLQPAPQDSGRS
CGVDFEVRAFATDSTDAEEDRIPRRSSVRLLIRRVQHAPLEMGPQPRAEAAWQFFMSDRPLHLAVSLNREIYF
HGEPIPVTVTVTNNTERTVRRIRAFVEQVANVVLYSSDYYVRPVAMEEAQERVPPNSTLTRTLTLLPLLANNR
ERRGIALDGRIRHEDTNLASSTIIREGIDRTVLGILVSYQIRVRLTVSGFLGELTSSEVATEVPFRLMHPQPG
GPARESYQDANLVFEEFARHNLRDAGEAEEGRRDRNDVDE
SEQ ID NO: 22. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTRLQESLLRRLGSNTYPFLLTFPDYLPCSVMLQPAPQDSGRS
CGVDFEVRAFATDSTDAEEDRIPRRSSVRLLIRRVQHAPLEMGPQPRAEAAWQFFMSDRPLHLAVSLNREIYF
HGEPIPVTVTVTNNTERTVRRIRAFVEQVANVVLYSSDYYVRPVAMEEAQERVPPNSTLTRTLTLLPLLANNR
ERRGIALDGRIRHEDTNLASSTIIREGIDRTVLGILVSYQIRVRLTVSGFLGELTSSEVATEVPFRLMHPQPA
APARESYQDANLVFEEFARHNLRDAGEAEEGRRDRNDVDE
SEQ ID NO: 23. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTKLQESLLKKLGSNTYPFLLTFPDYLPCSVMLQPAPQDSGKS
CGVDFEVRAFATDSTDAEEDRIPRRSSVRLLIRRVQHAPLEMGPQPRAEAAWQFFMSDRPLHLAVSLNREIYF
HGEPIPVTVTVTNNTEKTVKKIKAFVEQVANVVLYSSDYYVKPVAMEEAQEKVPPNSTLTKTLTLLPLLANNR
ERRGIALDGKIKHEDTNLASSTIIKEGIDRTVLGILVSYQIKVKLTVSGFLGELTSSEVATEVPFRLMHPQPA
GPARESYQDANLVFEEFARHNLRDAGEAEEGRRDRNDVDE
SEQ ID NO: 24. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTKLQESLLKKLGSNTYPFLLTFPDYLPCSVMLQPAPQDSGKS
CGVDFEVKAFATDSTDAEEDKIPKKSSVRLLIRKVQHAPLEMGPQPRAEAAWQFFMSDKPLHLAVSLNKEIYF
HGEPIPVTVTVTNNTEKTVKKIKAFVEQVANVVLYSSDYYVKPVAMEEAQEKVPPNSTLTKTLTLLPLLANNR
ERRGIALDGKIKHEDTNLASSTIIKEGIDRTVLGILVSYQIKVKLTVSGFLGELTSSEVATEVPFRLMHPQPG
APAKESYQDANLVFEEFARHNLKDAGEAEEGKRDKNDVDE
SEQ ID NO: 25. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTKLQESLLKKLGSNTYPFLLTFPDYLPCSVMLQPAPQDSGKS
CGVDFEVKAFATDSTDAEEDKIPKKSSVRLLIRKVQHAPLEMGPQPRAEAAWQFFMSDKPLHLAVSLNKEIYF
HGEPIPVTVTVTNNTEKTVKKIKAFVEQVANVVLYSSDYYVKPVAMEEAQEKVPPNSTLTKTLTLLPLLANNR
ERRGIALDGKIKHEDTNLASSTIIKEGIDRTVLGILVSYQIKVKLTVSGFLGELTSSEVATEVPFRLMHPQPA
DPARESYQDANLVFEEFARHNLRDAGEAEEGRRDRNDVDE
SEQ ID NO: 26. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTKLQESLLKKLGSNTYPFLLTFPDYLPCSVMLQPAPQDSGKS
CGVDFEVKAFATDSTDAEEDKIPKKSSVRLLIRKVQHAPLEMGPQPRAEAAWQFFMSDKPLHLAVSLNKEIYF
HGEPIPVTVTVTNNTERTVRRIRAFVEQVANVVLYSSDYYVRPVAMEEAQERVPPNSTLTRTLTLLPLLANNR
ERRGIALDGRIRHEDTNLASSTIIREGIDRTVLGILVSYQIRVRLTVSGFLGELTSSEVATEVPFRLMHPQPG
DPARESYQDANLVFEEFARHNLRDAGEAEEGRRDRNDVDE
SEQ ID NO: 27. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTRLQESLLRRLGSNTYPFLLTFPDYLPCSVMLQPAPQDSGRS
CGVDFEVRAFATDSTDAEEDRIPRRSSVRLLIRRVQHAPLEMGPQPRAEAAWQFFMSDRPLHLAVSLNREIYF
HGEPIPVTVTVTNNTEKTVKKIKAFVEQVANVVLYSSDYYVKPVAMEEAQEKVPPNSTLTKTLTLLPLLANNR
ERRGIALDGRIRHEDTNLASSTIIREGIDRTVLGILVSYQIRVRLTVSGFLGELTSSEVATEVPFRLMHPQPE
APARESYQDANLVFEEFARHNLRDAGEAEEGRRDRNDVDE
SEQ ID NO: 28. Human variant arrestin-1; amino acid sequence
MAASGKTSKSEPNHVIFKKISRDKSVTIYLGNRDYIDHVSQVQPVDGVVLVDPDLVKGKKVYVTLTCAFRYGQ
EDIDVIGLTFRRDLYFSRVQVYPPVGAASTPTRLQESLLRRLGSNTYPFLLTFPDYLPCSVMLQPAPQDSGRS
CGVDFEVRAFATDSTDAEEDRIPRRSSVRLLIRRVQHAPLEMGPQPRAEAAWQFFMSDRPLHLAVSLNREIYF
HGEPIPVTVTVTNNTEKTVKKIKAFVEQVANVVLYSSDYYVKPVAMEEAQEKVPPNSTLTKTLTLLPLLANNR
ERRGIALDGRIRHEDTNLASSTIIREGIDRTVLGILVSYQIRVRLTVSGFLGELTSSEVATEVPFRLMHPQPE
GPARESYQDANLVFEEFARHNLRDAGEAEEGRRDRNDVDE
SEQ ID NO: 29. bovine wild-type arrestin-1; nucleic acid sequence
atgaaggccaataagcccgcaccaaaccacgttatcttcaagaagatctcccgtgataaatcggtgaccatct
acctggggaagagagattacatagaccacgttgaacgagtagagcctgtggatggcgtcgtgctggtggatcc
tgagctcgtgaagggcaagagagtgtacgtgtctctgacgtgtgccttccgctacggccaggaagacatcgac
gtgatgggcctcagcttccgcagggacctctacttctcccaggtccaggtgttccctcccgtgggggcctcgg
gcgccaccacgaggctgcaggagagcctcatcaagaagctgggggccaacacctaccccttcctgctcacgtt
tcctgactacttgccctgttcggtgatgctgcagccagctccgcaagatgtgggcaagagctgtggggtcgac
tttgagatcaaagcattcgccacgcacagcacagatgtggaagaggacaaaattcccaagaagagctccgtgc
gtttgctgatccggaaggtacagcacgcgccacgcgatatgggtccccagccccgagccgaggcctcctggca
gttcttcatgtcggacaagcccctgcgcctcgccgtctcgctcagcaaagagatctattaccacggggaaccc
attcctgtgaccgtggccgtgaccaacagcacagagaagacagtgaagaagattaaagtgctagtggagcaag
tgaccaacgtggttctctactcgagtgattattacatcaagacggtggctgccgaggaagcacaggaaaaagt
gccgccaaacagctcgctgaccaagacgctgacgctggtgcccttgctggccaacaaccgtgagagaaggggc
atcgccctggatgggaaaatcaagcacgaggacacgaacctggcctccagcaccatcataaaggagggaatag
acaagaccgtcatggggatcctggtgtcttaccagatcaaggtgaagctcacggtgtcaggccttctgggaga
gctcacatccagtgaagtggccactgaggtgccgttccgcctcatgcatccccagccagaggacccagatacc
gccaaggaaagttttcaggatgaaaattttgtttttgaggagtttgctcgccaaaatctgaaagatgcaggag
aatataaggaagagaagacagaccaggaggcggctatggatgagtga
SEQ ID NO: 30. bovine wild-type arrestin-1; amino acid sequence
MKANKPAPNHVIFKKISRDKSVTIYLGKRDYIDHVERVEPVDGVVLVDPELVKGKRVYVSLTCAFRYGQEDID
VMGLSFRRDLYFSQVQVFPPVGASGATTRLQESLIKKLGANTYPFLLTFPDYLPCSVMLQPAPQDVGKSCGVD
FEIKAFATHSTDVEEDKIPKKSSVRLLIRKVQHAPRDMGPQPRAEASWQFFMSDKPLRLAVSLSKEIYYHGEP
IPVTVAVTNSTEKTVKKIKVLVEQVTNWLYSSDYYIKTVAAEEAQEKVPPNSSLTKTLTLVPLLANNRERRG
IALDGKIKHEDTNLASSTIIKEGIDKTVMGILVSYQIKVKLTVSGLLGELTSSEVATEVPFRLMHPQPEDPDT
AKESFQDENFVFEEFARQNLKDAGEYKEEKTDQEAAMDE

1. An arrestin-1 variant comprising an amino acid substitution of the glutamate residue and/or the aspartate residue corresponding to positions 365 and 366 of SEQ ID NO: 18 or an ortholog thereof, wherein the substitution at each position is independently alanine or glycine.

2. The arrestin-1 variant of claim 1, wherein the arrestin-1 variant contains an alanine or glycine substitution of the glutamate residue at position 365.

3. The arrestin-1 variant of claim 2, wherein the substitution is a glycine.

4. The arrestin-1 variant of claim 1, wherein the arrestin-1 variant contains an alanine substitution of the aspartate residue at position 366.

5. The arrestin-1 variant of claim 4, wherein the substitution is a glycine.

6. The arrestin-1 variant of claim 1, wherein the arrestin-1 variant contains substitutions of the glutamate residue at position 365 and the aspartate residue at position 366, wherein the substitution at each position is independently alanine or glycine.

7. The arrestin-1 variant of claim 6, wherein the variant comprises:
(a) alanine substitutions at positions 365 and 366,
(b) an alanine substitution at position 365 and a glycine substitution at position 362,
(c) a glycine substitution at position 365 and an alanine substitution at position 362, or
(d) glycine substitutions at positions 365 and 366.

8. The arrestin-1 variant of claim 1, wherein the arrestin-1 variant comprises the amino acid sequence of SEQ ID NO: 6, 9-16, or 21-28 or an ortholog thereof or an amino acid sequence having at least 70% identity to the amino acid sequence of SEQ ID NO: 6, 9-16, or 21-28.

9. The arrestin-1 variant of any one of claims 1-8, wherein the ortholog comprises a mammalian ortholog.

10. The arrestin-1 variant of claim 9, wherein the mammalian ortholog comprises a rat ortholog, a guinea pig ortholog, a rabbit ortholog, a non-human primate ortholog, dog ortholog, cat ortholog, or sheep ortholog.

11. The arrestin-1 variant of any one of claims 1-10, wherein the arrestin-1 variant has decreased inhibitory effect on enolase-1 catalytic activity compared to a wild-type arrestin-1 protein.

12. A nucleic acid encoding the arrestin-1 variant of any one of claims 1-11.

13. The nucleic acid of claim 12 wherein the nucleic acid comprises the sequence of SEQ ID NO: 17 or an ortholog thereof, wherein the sequence encoding the amino acids corresponding to positions 365 and 366 of SEQ ID NO: 18 is selected from the group consisting of: GCNGCN, GCNGGN, GGNGCN, GGNGGN, GCNGAR, GGNGAR, GAYGCN, and GAYGGN, wherein N is A, G, C or T, Y is C or T, and R is A or G.

14. The nucleic acid sequence of claim 13, wherein the nucleic acid comprises the sequence of SEQ ID NO: 5, 7, 8, 19, or 20, or a nucleic acid sequence having at least 70% identity to the sequence of SEQ ID NO: 5, 7, 8, 19, or 20.

15. A vector comprising a polynucleotide encoding the arrestin-1 variant of any one of claims 1-11.

16. The vector of claim 15, wherein the polynucleotide comprises the nucleic acid of any one of claims 12-14.

17. The vector of claim 16, wherein the vector comprises a plasmid.

18. The vector of claim 16, wherein the vector comprises a viral vector.

19. The vector of claim 18, wherein the vector comprises an adeno-associated virus (AAV) vector.

20. A method of treating a subject having a retinal degenerative disease or at risk of developing a retinal degenerative disease comprising administering to the eye of the subject the arrestin-1 variant of any one of claims 1-11, or a nucleic acid encoding the arrestin-1 variant.

21. The method of claim 20, wherein administering the arrestin-1 variant comprises administering the vector of any one of claims 15-19.

22. The method of claim 21, wherein the vector is an AAV vector and administering the AAV vector comprises subretinal injection, intravitreal injection, or suprachoroidal injection.

23. The method of any one of claims 20-22, wherein the retinal degenerative disease is associated with loss of rod and/or cone photoreceptors.

24. The method of claim 23, wherein the retinal degenerative disease is retinitis pigmentosa, a cone-rod dystrophy, or Usher's syndrome.

25. The method of any one of claims 20-25, wherein administering the arrestin-1 variant to the eye results in one or more of: increased survival of photoreceptors, improved photoreceptor function, decreased photoreceptor degeneration, decreased degeneration of rod cell and/or the cone cells, increased rate of glycolysis in rod cells and/or cone cells, improved scotopic and/or photopic vision and delay of onset of photoreceptor degeneration.


SEQUENCE LISTING (102 sequences)

SEQ ID NO: 1 - DNA, 1263 bp, Artificial Sequence
  Modified arrestin-1 sequence
atgcaccaccaccaccaccacaccatggcagcctgcgggaagaccaataaatcccacgtcatcttcaagaaggtctctcgggacaagtcggtgaccatctacctggggaagagagactatgtagaccacgtcagccaagtagagcctgtagatggtgttgtgctggtggaccctgagcttgtgaaggggaagaaggtgtatgtcaccctgacctgcgccttccgctatggccaggaggacattgatgtgatgggcctgaccttccgcagggacctgtatttttctcgggtccaggtataccctcctgttggggccatgagtgtcctcacccaactccaagagagcctgctcaagaaactgggagacaacacgtacccctttctgctcacgtttcctgactacctaccctgttcagtgatgttgcagccagctccccaggatgtaggcaagagctgtggggttgactttgaggttaaagcatttgcctcagacatcacagatcctgaggaggacaagatccccaagaagagctctgtgcggttactgatccggaaagtgcagcatgccccacctgagatgggtccccagcccagtgctgaagcctcctggcagttcttcatgtctgacaagcccctgaacctctcagtctccctcagcaaagagatatatttccatggggagcccatccccgtgactgtgaccgtgaccaacaacaccgacaaggttgtgaagaagattaaagtatcagtggaacagatagccaatgtggttctctactcaagtgactattacgtcaagcctgtagcctcggaggaaacacaagaaaaagtgcagccaaacagcactttgaccaagacactggtgctggtgcccctactggctaacaacagagaaagaagaggcatcgcgctggatgggaagatcaagcatgaggacacaaacctggcctccagcaccatcatcaaggagggcatcgaccggaccgtcatggggatcctggtgtcataccatatcaaggtgaagctcacggtgtcgggctttctaggagagctcacatccagtgaagtggctaccgaggtgccgttccgcctcatgcacccgcagcctgaggatccagcaaaagaaagtgttcaggatgaaaatttggtttttgaagagtttgctcgccaaaatctgaaagatactggagagaacacagaagggaagaaagatgaggatgctggccaggatgaggagcaaaaacttatctctgaggaagatctg

SEQ ID NO: 2 - protein, 421 aa, Artificial Sequence
  modified arrestin-1 sequence
MHHHHHHTMAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPEDPAKESVQDENLVFEEFARQNLKDTGENTEGKKDEDAGQDEEQKLISEEDL

SEQ ID NO: 3 - DNA, 1209 bp, Mus musculus
atggcagcctgcgggaagaccaataaatcccacgtcatcttcaagaaggtctctcgggacaagtcggtgaccatctacctggggaagagagactatgtagaccacgtcagccaagtagagcctgtagatggtgttgtgctggtggaccctgagcttgtgaaggggaagaaggtgtatgtcaccctgacctgcgccttccgctatggccaggaggacattgatgtgatgggcctgaccttccgcagggacctgtatttttctcgggtccaggtataccctcctgttggggccatgagtgtcctcacccaactccaagagagcctgctcaagaaactgggagacaacacgtacccctttctgctcacgtttcctgactacctaccctgttcagtgatgttgcagccagctccccaggatgtaggcaagagctgtggggttgactttgaggttaaagcatttgcctcagacatcacagatcctgaggaggacaagatccccaagaagagctctgtgcggttactgatccggaaagtgcagcatgccccacctgagatgggtccccagcccagtgctgaagcctcctggcagttcttcatgtctgacaagcccctgaacctctcagtctccctcagcaaagagatatatttccatggggagcccatccccgtgactgtgaccgtgaccaacaacaccgacaaggttgtgaagaagattaaagtatcagtggaacagatagccaatgtggttctctactcaagtgactattacgtcaagcctgtagcctcggaggaaacacaagaaaaagtgcagccaaacagcactttgaccaagacactggtgctggtgcccctactggctaacaacagagaaagaagaggcatcgcgctggatgggaagatcaagcatgaggacacaaacctggcctccagcaccatcatcaaggagggcatcgaccggaccgtcatggggatcctggtgtcataccatatcaaggtgaagctcacggtgtcgggctttctaggagagctcacatccagtgaagtggctaccgaggtgccgttccgcctcatgcacccgcagcctgaggatccagcaaaagaaagtgttcaggatgaaaatttggtttttgaagagtttgctcgccaaaatctgaaagatactggagagaacacagaagggaagaaagatgaggatgctggccaggatgag

SEQ ID NO: 4 - protein, 409 aa, Mus musculus
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPEDPAKESVQDENLVFEEFARQNLKDTGENTEGKKDEDAGQDEDAGQDE

SEQ ID NO: 5 - DNA, 1263 bp, Artificial Sequence
  modified arrestin-1 sequence
atgcaccaccaccaccaccacaccatggcagcctgcgggaagaccaataaatcccacgtcatcttcaagaaggtctctcgggacaagtcggtgaccatctacctggggaagagagactatgtagaccacgtcagccaagtagagcctgtagatggtgttgtgctggtggaccctgagcttgtgaaggggaagaaggtgtatgtcaccctgacctgcgccttccgctatggccaggaggacattgatgtgatgggcctgaccttccgcagggacctgtatttttctcgggtccaggtataccctcctgttggggccatgagtgtcctcacccaactccaagagagcctgctcaagaaactgggagacaacacgtacccctttctgctcacgtttcctgactacctaccctgttcagtgatgttgcagccagctccccaggatgtaggcaagagctgtggggttgactttgaggttaaagcatttgcctcagacatcacagatcctgaggaggacaagatccccaagaagagctctgtgcggttactgatccggaaagtgcagcatgccccacctgagatgggtccccagcccagtgctgaagcctcctggcagttcttcatgtctgacaagcccctgaacctctcagtctccctcagcaaagagatatatttccatggggagcccatccccgtgactgtgaccgtgaccaacaacaccgacaaggttgtgaagaagattaaagtatcagtggaacagatagccaatgtggttctctactcaagtgactattacgtcaagcctgtagcctcggaggaaacacaagaaaaagtgcagccaaacagcactttgaccaagacactggtgctggtgcccctactggctaacaacagagaaagaagaggcatcgcgctggatgggaagatcaagcatgaggacacaaacctggcctccagcaccatcatcaaggagggcatcgaccggaccgtcatggggatcctggtgtcataccatatcaaggtgaagctcacggtgtcgggctttctaggagagctcacatccagtgaagtggctaccgaggtgccgttccgcctcatgcacccgcagcctggaggtccagcaaaagaaagtgttcaggatgaaaatttggtttttgaagagtttgctcgccaaaatctgaaagatactggagagaacacagaagggaagaaagatgaggatgctggccaggatgaggagcaaaaacttatctctgaggaagatctg

SEQ ID NO: 6 - protein, 421 aa, Artificial Sequence
  modified arrestin-1 sequence
MHHHHHHTMAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPGGPAKESVQDENLVFEEFARQNLKDTGENTEGKKDEDAGQDEEQKLISEEDL

SEQ ID NO: 7 - DNA, 1209 bp, Artificial Sequence
  modified arrestin-1 sequence
atggcagcctgcgggaagaccaataaatcccacgtcatcttcaagaaggtctctcgggacaagtcggtgaccatctacctggggaagagagactatgtagaccacgtcagccaagtagagcctgtagatggtgttgtgctggtggaccctgagcttgtgaaggggaagaaggtgtatgtcaccctgacctgcgccttccgctatggccaggaggacattgatgtgatgggcctgaccttccgcagggacctgtatttttctcgggtccaggtataccctcctgttggggccatgagtgtcctcacccaactccaagagagcctgctcaagaaactgggagacaacacgtacccctttctgctcacgtttcctgactacctaccctgttcagtgatgttgcagccagctccccaggatgtaggcaagagctgtggggttgactttgaggttaaagcatttgcctcagacatcacagatcctgaggaggacaagatccccaagaagagctctgtgcggttactgatccggaaagtgcagcatgccccacctgagatgggtccccagcccagtgctgaagcctcctggcagttcttcatgtctgacaagcccctgaacctctcagtctccctcagcaaagagatatatttccatggggagcccatccccgtgactgtgaccgtgaccaacaacaccgacaaggttgtgaagaagattaaagtatcagtggaacagatagccaatgtggttctctactcaagtgactattacgtcaagcctgtagcctcggaggaaacacaagaaaaagtgcagccaaacagcactttgaccaagacactggtgctggtgcccctactggctaacaacagagaaagaagaggcatcgcgctggatgggaagatcaagcatgaggacacaaacctggcctccagcaccatcatcaaggagggcatcgaccggaccgtcatggggatcctggtgtcataccatatcaaggtgaagctcacggtgtcgggctttctaggagagctcacatccagtgaagtggctaccgaggtgccgttccgcctcatgcacccgcagcctggaggtccagcaaaagaaagtgttcaggatgaaaatttggtttttgaagagtttgctcgccaaaatctgaaagatactggagagaacacagaagggaagaaagatgaggatgctggccaggatgag

SEQ ID NO: 8 - DNA, 1209 bp, Artificial Sequence
  modified arrestin-1 sequence
  misc_feature (1086)..(1086): n is a, c, g, or t
  misc_feature (1089)..(1089): n is a, c, g, or t
atggcagcctgcgggaagaccaataaatcccacgtcatcttcaagaaggtctctcgggacaagtcggtgaccatctacctggggaagagagactatgtagaccacgtcagccaagtagagcctgtagatggtgttgtgctggtggaccctgagcttgtgaaggggaagaaggtgtatgtcaccctgacctgcgccttccgctatggccaggaggacattgatgtgatgggcctgaccttccgcagggacctgtatttttctcgggtccaggtataccctcctgttggggccatgagtgtcctcacccaactccaagagagcctgctcaagaaactgggagacaacacgtacccctttctgctcacgtttcctgactacctaccctgttcagtgatgttgcagccagctccccaggatgtaggcaagagctgtggggttgactttgaggttaaagcatttgcctcagacatcacagatcctgaggaggacaagatccccaagaagagctctgtgcggttactgatccggaaagtgcagcatgccccacctgagatgggtccccagcccagtgctgaagcctcctggcagttcttcatgtctgacaagcccctgaacctctcagtctccctcagcaaagagatatatttccatggggagcccatccccgtgactgtgaccgtgaccaacaacaccgacaaggttgtgaagaagattaaagtatcagtggaacagatagccaatgtggttctctactcaagtgactattacgtcaagcctgtagcctcggaggaaacacaagaaaaagtgcagccaaacagcactttgaccaagacactggtgctggtgcccctactggctaacaacagagaaagaagaggcatcgcgctggatgggaagatcaagcatgaggacacaaacctggcctccagcaccatcatcaaggagggcatcgaccggaccgtcatggggatcctggtgtcataccatatcaaggtgaagctcacggtgtcgggctttctaggagagctcacatccagtgaagtggctaccgaggtgccgttccgcctcatgcacccgcagcctggnggnccagcaaaagaaagtgttcaggatgaaaatttggtttttgaagagtttgctcgccaaaatctgaaagatactggagagaacacagaagggaagaaagatgaggatgctggccaggatgag

SEQ ID NO: 9 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPGGPAKESVQDENLVFEEFARQNLKDTGENTEGKKDEDAGQDE

SEQ ID NO: 10 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPAAPAKESVQDENLVFEEFARQNLKDTGENTEGKKDEDAGQDE

SEQ ID NO: 11 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPAGPAKESVQDENLVFEEFARQNLKDTGENTEGKKDEDAGQDE

SEQ ID NO: 12 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPGAPAKESVQDENLVFEEFARQNLKDTGENTEGKKDEDAGQDE

SEQ ID NO: 13 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPGDPAKESVQDENLVFEEFARQNLKDTGENTEGKKDEDAGQDE

SEQ ID NO: 14 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPADPAKESVQDENLVFEEFARQNLKDTGENTEGKKDEDAGQDE

SEQ ID NO: 15 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPEAPAKESVQDENLVFEEFARQNLKDTGENTEGKKDEDAGQDE

SEQ ID NO: 16 - protein, 403 aa, Artificial Sequence
  modified arrestin-1 sequence
MAACGKTNKSHVIFKKVSRDKSVTIYLGKRDYVDHVSQVEPVDGVVLVDPELVKGKKVYVTLTCAFRYGQEDIDVMGLTFRRDLYFSRVQVYPPVGAMSVLTQLQESLLKKLGDNTYPFLLTFPDYLPCSVMLQPAPQDVGKSCGVDFEVKAFASDITDPEEDKIPKKSSVRLLIRKVQHAPPEMGPQPSAEASWQFFMSDKPLNLSVSLSKEIYFHGEPIPVTVTVTNNTDKVVKKIKVSVEQIANVVLYSSDYYVKPVASEETQEKVQPNSTLTKTLVLVPLLANNRERRGIALDGKIKHEDTNLASSTIIKEGIDRTVMGILVSYHIKVKLTVSGFLGELTSSEVATEVPFRLMHPQPEGPAKESVQDENLVFEEFARQNLKDTGENTEGKKDEDAGQDE

SEQ ID NO: 17 - DNA, 1215 bp, Homo sapiens
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctgaggacccagctaaggaaagttatcaggatgcaaatttagtttttgaggagtttgctcgccataatctgaaagatgcaggagaagctgaggaggggaagagagacaagaatgacgttgatgag

SEQ ID NO: 18 - protein, 405 aa, Homo sapiens
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 19 - DNA, 1215 bp, Artificial Sequence
  modified arrestin-1 sequence
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctggaggtccagctaaggaaagttatcaggatgcaaatttagtttttgaggagtttgctcgccataatctgaaagatgcaggagaagctgaggaggggaagagagacaagaatgacgttgatgag

SEQ ID NO: 20 - DNA, 1215 bp, Artificial Sequence
  modified arrestin-1 sequence
  misc_feature (1095)..(1095): n is a, c, g, or t
  misc_feature (1098)..(1098): n is a, c, g, or t
atggcagccagcgggaagaccagcaagtccgaaccgaaccatgttatcttcaagaagatctcccgggacaaatcggtgaccatctacctggggaacagagactacatagaccatgtcagccaagtccagcctgtggatggtgtcgtgttggttgatcctgatcttgtgaagggaaagaaagtgtatgtcactctgacctgcgccttccgctatggccaagaggacattgacgtgatcggcttgaccttccgcagggacctgtacttctcccgggtccaggtgtatcctcctgtgggggccgcgagcacccccacaaaactgcaagagagcctgcttaaaaagctggggagcaacacgtacccctttctcctgacgtttcctgactacttgccctgttcagtgatgttgcagccagctccacaagattcagggaagtcctgtggggttgactttgaggtcaaagcattcgccacagacagcaccgatgccgaagaggacaaaatccccaagaagagctccgtgcgattactgatccgcaaagtacagcatgccccacttgagatgggtccccagccccgagctgaggcggcctggcagttcttcatgtctgacaagcccctgcaccttgcggtctctctcaacaaagagatctatttccatggggagcccatccctgtgaccgtgactgtcaccaataacacagagaagaccgtgaagaagattaaagcattcgtggaacaggtggccaatgtggttctctactcgagtgattattacgtcaagcccgtggctatggaggaagcgcaagaaaaagtgccaccaaacagcactttgaccaagacgctgacgctgctgcccttgctggctaacaatcgagaaaggagaggcattgccctggatgggaaaatcaagcacgaggacacaaaccttgcctccagcaccatcattaaggagggcatagaccggaccgtcctgggaatcctggtgtcttaccagatcaaggtgaagctcacagtgtcaggctttctgggagagctcacctccagtgaagtcgccactgaggtcccattccgcctcatgcaccctcagcctggnggnccagctaaggaaagttatcaggatgcaaatttagtttttgaggagtttgctcgccataatctgaaagatgcaggagaagctgaggaggggaagagagacaagaatgacgttgatgag

SEQ ID NO: 21 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPGGPAKESYQDANLVFEEFARHNLKDAGEAEEGKRDKNDVDE

SEQ ID NO: 22 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPAAPAKESYQDANLVFEEFARHNLKDAGEAEEGKRDKNDVDE

SEQ ID NO: 23 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPAGPAKESYQDANLVFEEFARHNLKDAGEAEEGKRDKNDVDE

SEQ ID NO: 24 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPGAPAKESYQDANLVFEEFARHNLKDAGEAEEGKRDKNDVDE

SEQ ID NO: 25 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPADPAKESYQDANLVFEEFARHNLKDAGEAEEGKRDKNDVDE

SEQ ID NO: 26 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPGDPAKESYQDANLVFEEFARHNLKDAGEAEEGKRDKNDVDE

SEQ ID NO: 27 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEAPAKESYQDANLVFEEFARHNLKDAGEAEEGKRDKNDVDE

SEQ ID NO: 28 - protein, 405 aa, Artificial Sequence
  modified arrestin-1 sequence
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEGPAKESYQDANLVFEEFARHNLKDAGEAEEGKRDKNDVDE

SEQ ID NO: 29 - DNA, 1215 bp, Bos taurus
atgaaggccaataagcccgcaccaaaccacgttatcttcaagaagatctcccgtgataaatcggtgaccatctacctggggaagagagattacatagaccacgttgaacgagtagagcctgtggatggcgtcgtgctggtggatcctgagctcgtgaagggcaagagagtgtacgtgtctctgacgtgtgccttccgctacggccaggaagacatcgacgtgatgggcctcagcttccgcagggacctctacttctcccaggtccaggtgttccctcccgtgggggcctcgggcgccaccacgaggctgcaggagagcctcatcaagaagctgggggccaacacctaccccttcctgctcacgtttcctgactacttgccctgttcggtgatgctgcagccagctccgcaagatgtgggcaagagctgtggggtcgactttgagatcaaagcattcgccacgcacagcacagatgtggaagaggacaaaattcccaagaagagctccgtgcgtttgctgatccggaaggtacagcacgcgccacgcgatatgggtccccagccccgagccgaggcctcctggcagttcttcatgtcggacaagcccctgcgcctcgccgtctcgctcagcaaagagatctattaccacggggaacccattcctgtgaccgtggccgtgaccaacagcacagagaagacagtgaagaagattaaagtgctagtggagcaagtgaccaacgtggttctctactcgagtgattattacatcaagacggtggctgccgaggaagcacaggaaaaagtgccgccaaacagctcgctgaccaagacgctgacgctggtgcccttgctggccaacaaccgtgagagaaggggcatcgccctggatgggaaaatcaagcacgaggacacgaacctggcctccagcaccatcataaaggagggaatagacaagaccgtcatggggatcctggtgtcttaccagatcaaggtgaagctcacggtgtcaggccttctgggagagctcacatccagtgaagtggccactgaggtgccgttccgcctcatgcatccccagccagaggacccagataccgccaaggaaagttttcaggatgaaaattttgtttttgaggagtttgctcgccaaaatctgaaagatgcaggagaatataaggaagagaagacagaccaggaggcggctatggatgagtga

SEQ ID NO: 30 - protein, 404 aa, Bos taurus [sequence omitted]

SEQ ID NO: 31 - DNA, 33 bp, Artificial Sequence
  Synthetic oligonucleotide
aagcccgcaccaaactgtgttatcttcaagaag

SEQ ID NO: 32 - DNA, 33 bp, Artificial Sequence
  Synthetic oligonucleotide
cttcttgaagataacacagtttggtgcgggctt

SEQ ID NO: 33 - DNA, 33 bp, Artificial Sequence
  Synthetic oligonucleotide
ttcaagaagatctcctgtgataaatcggtgacc

SEQ ID NO: 34 - DNA, 33 bp, Artificial Sequence
  Synthetic oligonucleotide
ggtcaccgatttatcacaggagatcttcttgaa

SEQ ID NO: 35 - DNA, 39 bp, Artificial Sequence
  Synthetic oligonucleotide
gataaatcggtgaccatctgtctggggaagagagattac

SEQ ID NO: 36 - DNA, 39 bp, Artificial Sequence
  Synthetic oligonucleotide
gtaatctctcttccccagacagatggtcaccgatttatc

SEQ ID NO: 37 - DNA, 34 bp, Artificial Sequence
  Synthetic oligonucleotide
gaccatctacctggggtgtagagattacatagac

SEQ ID NO: 38 - DNA, 34 bp, Artificial Sequence
  Synthetic oligonucleotide
gtctatgtaatctctacaccccaggtagatggtc

SEQ ID NO: 39 - DNA, 34 bp, Artificial Sequence
  Synthetic oligonucleotide
atagaccacgttgaatgtgtagagcctgtggatg

SEQ ID NO: 40 - DNA, 34 bp, Artificial Sequence
  Synthetic oligonucleotide
catccacaggctctacacattcaacgtggtctat

SEQ ID NO: 41 - DNA, 33 bp, Artificial Sequence
  Synthetic oligonucleotide
gtgctggtggatccatgtctcgtgaagggcaag

SEQ ID NO: 42 - DNA, 33 bp, Artificial Sequence
  Synthetic oligonucleotide
cttgcccttcacgagacatggatccaccagcac

SEQ ID NO: 43 - DNA, 33 bp, Artificial Sequence
  Synthetic oligonucleotide
gatcctgagctcgtgtgtggcaagagagtgtac

SEQ ID NO: 44 - DNA, 33 bp, Artificial Sequence
  Synthetic oligonucleotide
gtacactctcttgccacacacgagctcaggatc

SEQ ID NO: 45 - DNA, 30 bp, Artificial Sequence
  Synthetic oligonucleotide
tacggccaggaagactgtgacgtgatgggc

SEQ ID NO: 46 - DNA, 30 bp, Artificial Sequence
  Synthetic oligonucleotide
gcccatcacgtcacagtcttcctggccgta

SEQ ID NO: 47 - DNA, 35 bp, Artificial Sequence
  Synthetic oligonucleotide
cagggacctctacttctgtcaagtccaggtgttcc

SEQ ID NO: 48 - DNA, 35 bp, Artificial Sequence
  Synthetic oligonucleotide
ggaacacctggacttgacagaagtagaggtccctg

SEQ ID NO: 49 - DNA, 33 bp, Artificial Sequence
  Synthetic oligonucleotide
caggtgttccctccatgtggggcctcgggcgcc

SEQ ID NO: 50 - DNA, 33 bp, Artificial Sequence
  Synthetic oligonucleotide
ggcgcccgaggccccacatggagggaacacctg

SEQ ID NO: 51 - DNA, 33 bp, Artificial Sequence
  Synthetic oligonucleotide
atcaagaagctgggctgtaacacctaccccttc

SEQ ID NO: 52 - DNA, 33 bp, Artificial Sequence
  Synthetic oligonucleotide
gaaggggtaggtgttacagcccagcttcttgat

SEQ ID NO: 53 - DNA, 33 bp, Artificial Sequence
  Synthetic oligonucleotide
ctcacgtttcctgactgtttgccctgttcggtg

SEQ ID NO: 54 - DNA, 33 bp, Artificial Sequence
  Synthetic oligonucleotide
caccgaacagggcaaacagtcaggaaacgtgag

SEQ ID NO: 55 - DNA, 33 bp, Artificial Sequence
  Synthetic oligonucleotide
ccagctccgcaagattgtggcaagagcgcaggg

SEQ ID NO: 56 - DNA, 33 bp, Artificial Sequence
  Synthetic oligonucleotide
ccctgcgctcttgccacaatcttgcggagctgg

SEQ ID NO: 57 - DNA, 32 bp, Artificial Sequence
  Synthetic oligonucleotide
gaggacaaaattccctgtaagagctccgtgcg

SEQ ID NO: 58 - DNA, 32 bp, Artificial Sequence
  Synthetic oligonucleotide
cgcacggagctcttacagggaattttgtcctc

SEQ ID NO: 59 - DNA, 35 bp, Artificial Sequence
  Synthetic oligonucleotide
cagcacgcgccacgatgtatgggtccccagccccg

SEQ ID NO: 60 - DNA, 35 bp, Artificial Sequence
  Synthetic oligonucleotide
cggggctggggacccatacatcgtggcgcgtgctg

SEQ ID NO: 61 - DNA, 36 bp, Artificial Sequence
  Synthetic oligonucleotide
gatatgggtccccagccctgtgccgaggcctcctgg

SEQ ID NO: 62 - DNA, 36 bp, Artificial Sequence
  Synthetic oligonucleotide
ccaggaggcctcggcacagggctggggacccatatc

SEQ ID NO: 63 - DNA, 33 bp, Artificial Sequence
  Synthetic oligonucleotide
cgagccgaggcctcctgtcagttcttcatgtcg

SEQ ID NO: 64 - DNA, 33 bp, Artificial Sequence
  Synthetic oligonucleotide
cgacatgaagaactgacaggaggcctcggctcg

SEQ ID NO: 65 - DNA, 33 bp, Artificial Sequence
  Synthetic oligonucleotide
tggcagttcttcatgtgtgacaagcccctgcgc

SEQ ID NO: 66 - DNA, 33 bp, Artificial Sequence
  Synthetic oligonucleotide
gcgcaggggcttgtcacacatgaagaactgcca

SEQ ID NO: 67 - DNA, 33 bp, Artificial Sequence
  Synthetic oligonucleotide
ctcgccgtctcgctctgtaaagagatctattac

SEQ ID NO: 68 - DNA, 33 bp, Artificial Sequence
  Synthetic oligonucleotide
gtaatagatctctttacagagcgagacggcgag

SEQ ID NO: 69 - DNA, 33 bp, Artificial Sequence
  Synthetic oligonucleotide
atctattaccacgggtgtcccattcctgtgacc

SEQ ID NO: 70 - DNA, 33 bp, Artificial Sequence
  Synthetic oligonucleotide
ggtcacaggaatgggacacccgtggtaatagat

SEQ ID NO: 71 - DNA, 33 bp, Artificial Sequence
  Synthetic oligonucleotide
gtgaccaacagcacatgtaagacagtgaagaag

SEQ ID NO: 72 - DNA, 33 bp, Artificial Sequence
  Synthetic oligonucleotide
cttcttcactgtcttacatgtgctgttggtcac

SEQ ID NO: 73 - DNA, 28 bp, Artificial Sequence
  Synthetic oligonucleotide
cgtggttctctactgtagtgattattac

SEQ ID NO: 74 - DNA, 28 bp, Artificial Sequence
  Synthetic oligonucleotide
gtaataatcactacagtagagaaccacg

SEQ ID NO: 75 - DNA, 33 bp, Artificial Sequence
  Synthetic oligonucleotide
gaggaagcacaggaatgtgtgccgccaaacagc

SEQ ID NO: 76 - DNA, 33 bp, Artificial Sequence
  Synthetic oligonucleotide
gctgtttggcggcacacattcctgtgcttcctc

SEQ ID NO: 77 - DNA, 33 bp, Artificial Sequence
  Synthetic oligonucleotide
aagacgctgacgctgtgtcccttgctggccaac

SEQ ID NO: 78 - DNA, 33 bp, Artificial Sequence
  Synthetic oligonucleotide
gttggccagcaagggacacagcgtcagcgtctt

SEQ ID NO: 79 - DNA, 33 bp, Artificial Sequence
  Synthetic oligonucleotide
gggaaaatcaagcactgtgacacgaacctggcc

SEQ ID NO: 80 - DNA, 33 bp, Artificial Sequence
  Synthetic oligonucleotide
ggccaggttcgtgtcacagtgcttgattttccc

SEQ ID NO: 81 - DNA, 34 bp, Artificial Sequence
  Synthetic oligonucleotide
cataaaggagggaatatgtaagaccgtcatgggg

SEQ ID NO: 82 - DNA, 34 bp, Artificial Sequence
  Synthetic oligonucleotide
ccccatgacggtcttacatattccctcctttatg

SEQ ID NO: 83 - DNA, 33 bp, Artificial Sequence
  Synthetic oligonucleotide
catccccagccagagtgtccagataccgccaag

SEQ ID NO: 84 - DNA, 33 bp, Artificial Sequence
  Synthetic oligonucleotide
cttggcggtatctggacactctggctggggatg

SEQ ID NO: 85 - DNA, 33 bp, Artificial Sequence
  Synthetic oligonucleotide
gcaggagaatataagtgtgagaagacagaccag

SEQ ID NO: 86 - DNA, 33 bp, Artificial Sequence
  Synthetic oligonucleotide
ctggtctgtcttctcacacttatattctcctgc

SEQ ID NO: 87 - DNA, 34 bp, Artificial Sequence
  Synthetic oligonucleotide
catctacctggggaaggaagattacatagaccac

SEQ ID NO: 88 - DNA, 34 bp, Artificial Sequence
  Synthetic oligonucleotide
gtggtctatgtaatcttccttccccaggtagatg

SEQ ID NO: 89 - DNA, 36 bp, Artificial Sequence
  Synthetic oligonucleotide
gattacatagaccacgttaaacgagtagagcctgtg

SEQ ID NO: 90 - DNA, 36 bp, Artificial Sequence
  Synthetic oligonucleotide
cacaggctctactcgtttaacgtggtctatgtaatc

SEQ ID NO: 91 - DNA, 35 bp, Artificial Sequence
  Synthetic oligonucleotide
catagaccacgttgaagatgtagagcctgtggatg

SEQ ID NO: 92 - DNA, 35 bp, Artificial Sequence
  Synthetic oligonucleotide
catccacaggctctacatcttcaacgtggtctatg

SEQ ID NO: 93 - DNA, 32 bp, Artificial Sequence
  Synthetic oligonucleotide
cagcacgcgccacgcaagatgggtccccagcc

SEQ ID NO: 94 - DNA, 32 bp, Artificial Sequence
  Synthetic oligonucleotide
ggctggggacccatcttgcgtggcgcgtgctg

SEQ ID NO: 95 - DNA, 33 bp, Artificial Sequence
  Synthetic oligonucleotide
gggaaaatcaagcacaaggacacgaacctggcc

SEQ ID NO: 96 - DNA, 33 bp, Artificial Sequence
  Synthetic oligonucleotide
ggccaggttcgtgtccttgtgcttgattttccc

SEQ ID NO: 97 - DNA, 34 bp, Artificial Sequence
  Synthetic oligonucleotide
catgcatccccagccaaaggacccagataccgcc

SEQ ID NO: 98 - DNA, 34 bp, Artificial Sequence
  Synthetic oligonucleotide
ggcggtatctgggtcctttggctggggatgcatg

SEQ ID NO: 99 - DNA, 33 bp, Artificial Sequence
  Synthetic oligonucleotide
catccccagccagagaagccagataccgccaag

SEQ ID NO: 100 - DNA, 33 bp, Artificial Sequence
  Synthetic oligonucleotide
cttggcggtatctggcttctctggctggggatg

SEQ ID NO: 101 - DNA, 38 bp, Artificial Sequence
  Synthetic oligonucleotide
catgcatccccagccaggaggtccagataccgccaagg

SEQ ID NO: 102 - DNA, 38 bp, Artificial Sequence
  Synthetic oligonucleotide
ccttggcggtatctggacctcctggctggggatgcatg